(19) United States
(12) Patent Application Publication
(10) Pub. No.: US 2023/0078120 A1
(43) Pub. Date: Mar. 16, 2023

(54) Methods for Treating Coronavirus Infections

(71) Applicants: ASCENTAGE PHARMA (SUZHOU) CO.,LTD., SUZHOU (CN); ASCENTAGE PHARMA GROUP CORP LIMITED, CENTRAL (CN); GUANGZHOU HEALTHQUEST PHARMA CO., LTD., GUANGZHOU (CN)

(72) Inventors: Yifan Zhai, SUZHOU (CN); Dajun YANG, SUZHOU (CN); Douglas Dong FANG, SUZHOU (CN); Hengrui ZHU, SUZHOU (CN)

(21) Appl. No.: 17/778,664

(22) PCT Filed: Apr. 29, 2021
(86) PCT No.: PCT/CN2021/091169

§ 371 Date: May 20, 2022

Related U.S. Application Data
(60) Provisional application No. 63/019,508, filed on May 4, 2020.

(51) Int. Cl. A61K 31/496; A61K 31/407; A61K 31/675; A61K 31/635; A61P 31/14

(52) U.S. Cl. CPC A61K 31/496; A61K 31/407; A61K 31/675; A61K 31/635; A61P 31/14

(57) ABSTRACT

A method for treating patients with viral infections, particularly coronavirus infections, wherein the method comprises administering to the patient a therapeutically effective amount of a compound of any one of formulas (I)-(VI) or Compound 1, 2, 3, 4, or 5, or a pharmaceutically acceptable salt thereof.

### FIELD OF THE INVENTION

The present invention relates to methods for treating patients with coronavirus infections, including patients who are suffering from coronavirus disease 2019 (COVID-19).

### BACKGROUND OF THE INVENTION

Coronaviruses are a large family of viruses characterized by club-like spikes that project from their surface, an unusually large RNA genome, and a unique replication strategy. Fehr et al, Methods Mol. Biol., 2015, 1282: 1-23. Coronaviruses usually cause a variety of diseases in mammals and birds ranging from mild to moderate upper-respiratory tract illness, including illness caused by coronaviruses 229E, NL63, OC43, and HKUl. Common Human Coronaviruses, Centers for Disease Control and Prevention, available at https://www.cdc.gov/coronavirus/general-information.html (last visited Apr. 27, 2020). Over the past two decades, however, new coronaviruses have emerged from animal reservoirs to cause serious and widespread illness and death in humans. Coronaviruses, National Institute of Allergy and Infectious Diseases, available at https://wwv.niaid.nih.gov/diseases-conditions/coronaviruses (last visited Apr. 27, 2020).

In 2002 and 2003, the outbreaks of the highly pathogenic Severe Acute Respiratory Syndrome Coronavirus (SARS-CoV) caused over 8,000 cases worldwide with a death rate of ˜10%. In 2012, the Middle Eastern Respiratory Syndrome Coronavirus (MERS-CoV) caused 2,500 confirmed cases with a fatality rate of 36%. Wit et al., Nature Reviews Microbiology, 2016, 14, 523-534.

In 2019, a novel coronavirus called SARS-CoV-2 emerged and caused acute respiratory syndrome in humans called coronavirus disease 2019 (COVID-19). The World Health Organization declared a global pandemic on Mar. 11, 2020. Coronaviruses, National Institute of Allergy and Infectious Diseases, available at https://www.niaid.nih.gov/diseases-conditions/coronaviruses (last visited Apr. 27, 2020). Up to Apr. 26, 2020, there are a total of 2,992,970 confirmed COVID-19 cases and 207,518 confirmed deaths caused by SARS-CoV-2. John Hopkins University Coronavirus Resource Center, available at https://coronavirus.jhu.edu/map.html (last updated Apr. 27, 2020, 10:31:17 AM).

Treatment for coronavirus infection especially COVID-19 is in urgent need. Up to now, however, there are no clinically available antiviral drugs that can effectively treat diseases caused by SARS-CoV, SARS-CoV-2 or MERS-CoV. An efficient approach to drug discovery is to test existing antiviral drugs or drug candidates for new indications. Given the development times and manufacturing requirements for new products, repurposing of existing drugs or drug candidates is an attractive solution for outbreaks due to emerging viruses.

### SUMMARY OF THE INVENTION

In one aspect, the present invention relates to a method for treating a viral infection in a patient, comprising administering to the patient a therapeutically effective amount of Compound 1:

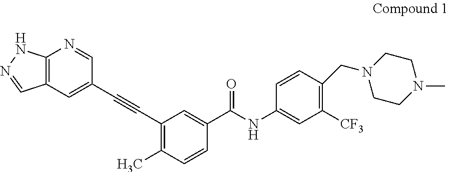 [SMILES: Cc1ccc(C(=O)Nc2ccc(CN3CCN(C)CC3)c(C(F)(F)F)c2)cc1C#Cc1cnc2[nH]ncc2c1]

or a pharmaceutically acceptable salt thereof.

In another aspect, the present invention relates to a method for treating a viral infection in a patient, comprising administering to the patient a therapeutically effective amount of Compound 2:

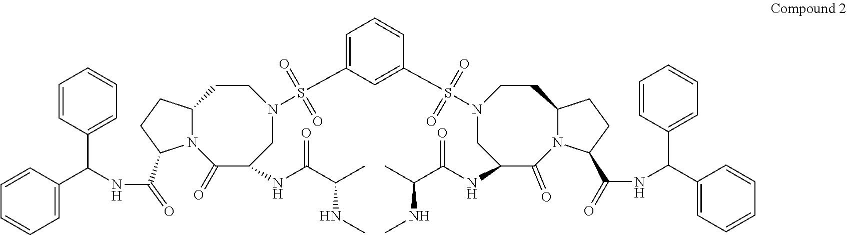 [SMILES: CN[C@@H](C)C(=O)N[C@H]1CN(S(=O)(=O)c2cccc(S(=O)(=O)N3CC[C@H]4CC[C@@H](C(=O)NC(c5ccccc5)c5ccccc5)N4C(=O)[C@@H](NC(=O)[C@H](C)NC)C3)c2)CC[C@H]2CC[C@@H](C(=O)NC(c3ccccc3)c3ccccc3)N2C1=O]

or a pharmaceutically acceptable salt thereof.

In another aspect, the present invention relates to a method for treating a viral infection in a patient, comprising administering to the patient a therapeutically effective amount of Compound 3:

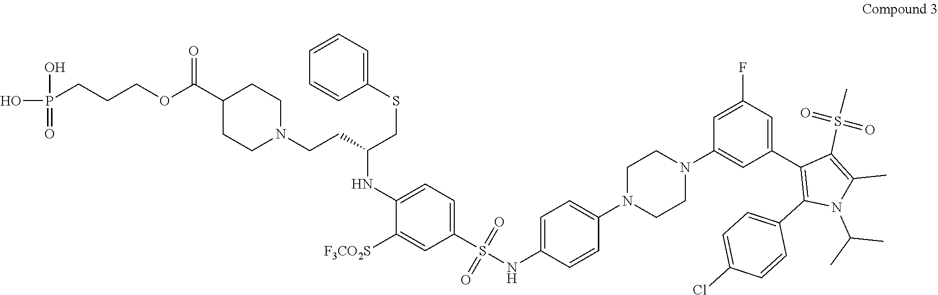

or a pharmaceutically acceptable salt thereof.

In another aspect, the present invention relates methods for treating a viral infection in a patient, comprising administering to the patient a therapeutically effective amount of Compound 4:

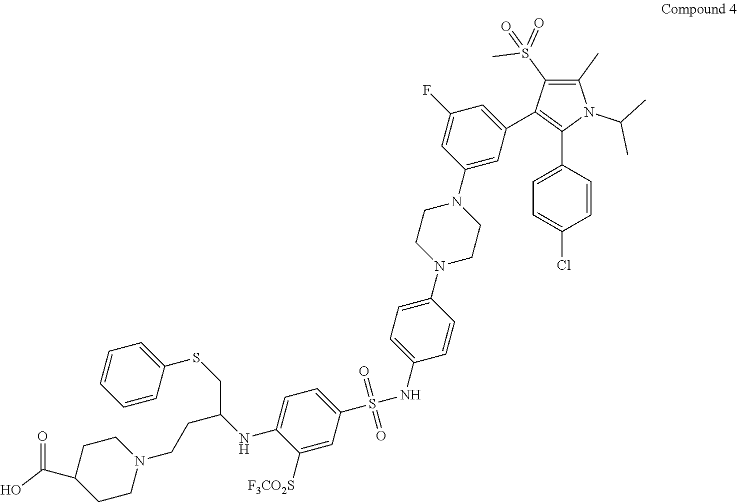

or a pharmaceutically acceptable salt thereof.

In another aspect, the present invention relates to a method for treating a viral infection in a patient, comprising administering to the patient a therapeutically effective amount of Compound 5:

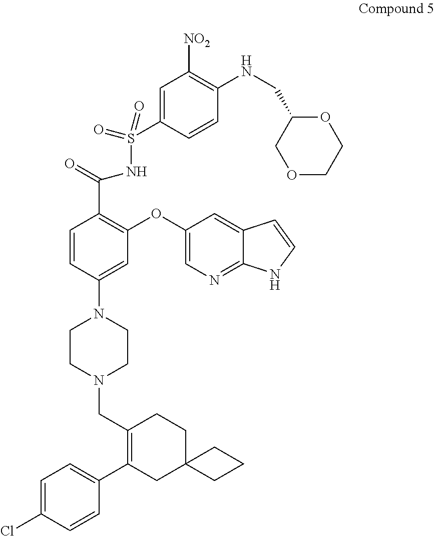 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt thereof.

In certain embodiments, the viral infection is caused by a coronavirus.

In certain embodiments, the viral infection is coronavirus infection caused by a coronavirus.

In certain embodiments, the coronavirus infection is caused by SARS-CoV-2, SARS-CoV, MERS-CoV, 229E, NL63, OC43, or HKUl.

In certain embodiments, the coronavirus infection is caused by SARS-CoV-2.

In certain embodiments, the coronavirus infection is caused by SARS-CoV.

In certain embodiments, the coronavirus infection is caused by MERS-CoV.

In certain embodiments, the present invention provides a method for treating a patient suffering from COVID-19.

In certain embodiments, the COVID-19 is caused by SARS-CoV-2.

In certain embodiments, the present invention provides a medicament or pharmaceutical composition comprising Compound 1 or a pharmaceutically acceptable salt thereof for treating a viral infection, particularly a coronavirus infection.

In certain embodiments, the present invention provides a medicament or pharmaceutical composition comprising Compound 2 or a pharmaceutically acceptable salt thereof for treating a viral infection, particularly a coronavirus infection.

In certain embodiments, the present invention provides a medicament or pharmaceutical composition comprising Compound 3 or a pharmaceutically acceptable salt thereof for treating a viral infection, particularly a coronavirus infection.

In certain embodiments, the present invention provides a medicament or pharmaceutical composition comprising Compound 4 or a pharmaceutically acceptable salt thereof for treating a viral infection, particularly a coronavirus infection.

In certain embodiments, the present invention provides a medicament or pharmaceutical composition comprising Compound 5 or a pharmaceutically acceptable salt thereof for treating a viral infection, particularly a coronavirus infection.

In certain embodiments, the present invention relates to use of Compound 1, or a pharmaceutically acceptable salt thereof in the manufacture of medicament for treating a viral infection, particularly a coronavirus infection.

In certain embodiments, the present invention relates to use of Compound 2, or a pharmaceutically acceptable salt thereof in the manufacture of medicament for treating a viral infection, particularly a coronavirus infection.

In certain embodiments, the present invention relates to use of Compound 3, or a pharmaceutically acceptable salt thereof in the manufacture of medicament for treating a viral infection, particularly a coronavirus infection.

In certain embodiments, the present invention relates to use of Compound 4, or a pharmaceutically acceptable salt thereof in the manufacture of medicament for treating a viral infection, particularly a coronavirus infection.

In certain embodiments, the present invention relates to use of Compound 5, or a pharmaceutically acceptable salt thereof in the manufacture of medicament for treating a viral infection, particularly a coronavirus infection.

### BRIEF DESCRIPTION OF THE FIGURES

FIG. 1 shows the effect of Compound 2 on apoptotic induction, IAPs expression, and HBV replication and gene expression in HepG2.2.15 liver cancer cell line.

FIG. 2 indicates Compound 2 promotes virus clearance in pAAV-HBV 1.3 model in C57BL/6J mice.

FIG. 3 shows that Compound 2 specifically induced apoptosis in the HBV-infected (HBsAg+) hepatocytes of HBV models in mice.

FIG. 4 shows that Compound 2 promotes the expression of IFN-γ and TNF-α, and the number of CD4+ and CD8+ T cells in liver tissue of HBV models.

FIG. 5 shows the procedure to test anti-viral activity of Compound 1 and Compound 2 using SARS-CoV-2 immunofluorescence assay (IFA). Imatinib, Remdesivir, Chloroquine, and Lopinavir were used as references.

FIG. 6 shows the results of anti-viral activity of Compound 1 and Compound 2 using SARS-CoV-2 immunofluorescence assay (IFA). Imatinib, Remdesivir, Chloroquine, and Lopinavir were used as references.

FIG. 7 shows the method of inhibition of cytokines associated with influenza virus induced cytokine storm.

FIG. 8 shows the results of inhibition of cytokines associated with influenza virus induced cytokine storm.

FIG. 9 shows the survival of influenza virus (IFV) infected mice treated with Compound 1 or reference compounds (Ponatinib, oseltamivir).

FIG. 10 shows the result of Compound 1 and ponatinib in the RIPK1 kinase assay. GSK2982772 was used as the reference.

### DETAILED DESCRIPTION OF THE INVENTION

All published documents cited herein are hereby incorporated herein by reference in their entirety.

Unless defined otherwise, all technical and scientific terms used herein have the same meaning as commonly understood by one of ordinary skill in the art to which this disclosure belongs.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

The term “comprises” refers to “includes, but is not limited to.”

As used herein, the terms “treatment,” “treat,” and “treating” refer to reversing, alleviating, delaying the onset of, or inhibiting the progress of a disease or disorder, or one or more symptoms thereof, including but not limited to therapeutic benefit. In some embodiments, treatment is administered after one or more symptoms have developed. In some embodiments, treatment may be administered in the absence of symptoms. For example, treatment may be administered to a subject prior to the onset of symptoms (e.g., in light of a history of symptoms and/or in light of genetic or other susceptibility factors). Treatment may also be continued after symptoms have resolved, for example to prevent or delay their recurrence.

Therapeutic benefit includes eradication and/or amelioration of the underlying disorder being treated such as cancer; it also includes the eradication and/or amelioration of one or more of the symptoms associated with the underlying disorder such that an improvement is observed in the subject, notwithstanding that the subject may still be afflicted with the underlying disorder. In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life.)

As used herein, “administering” or “administration” of the compound of formula (I) or formula (I-A) or a pharmaceutically acceptable salt thereof encompasses the delivery to a patient a compound or a pharmaceutically acceptable salt thereof, or a prodrug or other pharmaceutically acceptable derivative thereof, using any suitable formulation or route of administration, e.g., as described herein.

As used herein, the term “therapeutically effective amount” or “effective amount” refers to an amount that is effective to elicit the desired biological or medical response, including the amount of a compound that, when administered to a subject for treating a disorder, is sufficient to effect such treatment of the disorder. The effective amount will vary depending on the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

As used herein, “patient” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys).

As used herein, “pharmaceutically acceptable” or “physiologically acceptable” refer to compounds, salts, compositions, dosage forms and other materials which are useful in preparing a pharmaceutical composition that is suitable for veterinary or human pharmaceutical use.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of Compound 1 include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Although pharmaceutically acceptable counter ions will be preferred for preparing pharmaceutical formulations, other anions are quite acceptable as synthetic intermediates. Thus it may be pharmaceutically undesirable anions, such as iodide, oxalate, trifluoromethanesulfonate and the like, when such salts are chemical intermediates.

The term “pharmaceutically acceptable carrier” is used herein to refer to a material that is compatible with a recipient subject, preferably a mammal, more preferably a human, and is suitable for delivering an active agent to the target site without terminating the activity of the agent. The toxicity or adverse effects, if any, associated with the carrier preferably are commensurate with a reasonable risk/benefit ratio for the intended use of the active agent.

The term “orally” refers to administering a composition that is intended to be ingested. Examples of oral forms include, but are not limited to, tablets, pills, capsules, powders, granules, solutions or suspensions, and drops. Such forms may be swallowed whole or may be in chewable form.

The term “alkyl” means a branched-chain or straight chain alkyl group with the certain number of carbon atoms. For example, the definition of “C₁-C₅” in “C₁-C₅ alkyl” means straight-chain or branched-chain alkyl group with 1, 2, 3, 4 or 5 carbon atoms. For example, “C₁-C₅ alkyl” includes methyl, ethyl, n-propyl, isopropyl, n-butyl, tert-butyl, isobutyl, pentyl, etc.

The term “cycloalkyl” refers to a specific single saturated ring alkyl with the certain number of carbon atoms. For examples, “cycloalkyl” includes cyclopropyl-, methyl-cyclopropyl-, 2,2-dimethyl-cyclobutyl, 2-ethyl-cyclopentyl-, cyclohexyl etc.

The term of “Heterocycle” refers to an aromatic or nonaromatic ring containing 5-6 atoms, in which contains 1-4 hetero atoms such as O, N, S. “Heterocycle” includes hetero aromatic ring as mentioned above; it also includes dihydro and tetrahydro analogs. “Heterocycles” further includes but not limited to: imidazolyl, indolyl, isothiazolyl, isoxazolyl, oxadiazolyl, oxazolyl, oxetanyl, pyranyl, pyrazinyl, pyrazolyl, pyridazinyl, pyridyl, pyrimidinyl, pyrrolyl, quinazolinyl, tetrazolyl, thiadiazolyl, thiazolyl, thienyl, triazolyl, 1,4-alkyl-dioxinyl, alkyl pyrrolidinyl, dihydro-imidazolyl, dihydro-isoxazolyl, dihydro-iso thiazolyl, dihydro-oxadiazolyl, dihydro-oxazolyl, dihydro-pyrazinyl, dihydro-pyrazolyl, dihydro-pyridyl, dihydro-pyrimidinyl, dihydro-pyrrolyl, dihydro-tetrazolyl, dihydro-thiadiazolyl, dihydro-thiazolyl, dihydro-thienyl, dihydro-triazolyl, methylene dioxy-benzophenone acyl, tetrahydrofuranyl, tetrahydrothiophenyl, and their N-oxides etc. The linkage of heterocycle substituent can be achieved through C atom or heteroatom. In one embodiment, heterocycle is selected from imidazolyl, pyridyl, I-pyrrolidone, 2-piperidone, 2-pyrimidone, 2-pyrrolidone, thienyl, oxazolyl, triazolyl, isoxazolyl, etc.

The term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four, groups including, for example, halo, alkyl, alkenyl, —OCF₃, —CF₃, —NO₂, —CN, —NC, —OH, alkoxy, amino, alkylamino, —CO₂H, —CO₂ alkyl, —OCOalkyl, aryl, and heteroaryl.

The term of“heteroaryl” is a stable monocyclic ring with up to six atoms or a stable bicyclic ring in which each ring contains up to six atoms. At least one of the rings is an aromatic ring containing 1-4 atoms selected from O, N or S. Hetero aryl groups includes but not limited to: imidazolyl, triazolyl, pyrazolyl, furanyl, thienyl, oxazolyl, isoxazolyl, pyrazinyl, pyridazinyl, pyridyl, pyrimidinyl, pyrrolyl. About the definition of hetero aryl, any hetero aryl N-oxidation derivatives containing N atom should also be added. When hetero aryl substituted group is a bicyclic ring and one of the two rings is non-aromatic or non-hetero-atom containing ring, this bicyclic ring is fused through the aromatic ring or hetero atoms.

The term “halo” or “halogen” means chlorine, fluorine, bromine and iodine.

In one aspect, the disclosure relates to a method of treating a viral infection including a coronavirus infection in a human in need thereof, comprising administering to the patient a therapeutically effective amount of a compound of formula (I):

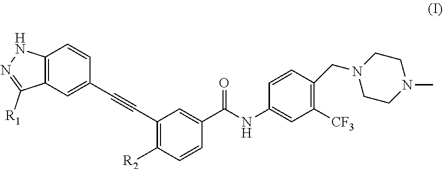

or a pharmaceutically acceptable salt thereof, wherein

R₁ is hydrogen, C₁₋₄ alkyl, C₃₋₆ cycloalkyl, C₁₋₄ alkyloxy, or phenyl; and

R₂ is hydrogen, C₁₋₄ alkyl, C₃₋₆ cycloalkyl, or halogen.

In one embodiment of the compound of formula (I), R₁ is hydrogen or C₁₋₄ alkyl.

In another embodiment of the compound of formula (I), R₁ is hydrogen.

In another embodiment of the compound of formula (I), R₂ is hydrogen or C₁₋₄ alkyl.

In another embodiment of the compound of formula (I), R₂ is C₁₋₄ alkyl.

In another embodiment of the compound of formula (I). R₂ is methyl or ethyl. In another embodiment, R₂ is methyl.

In another embodiment of the compound of formula (I), R₁ is hydrogen and R₂ is C₁₋₄ alkyl.

In one embodiment, the compound of formula (I) is Compound 1:

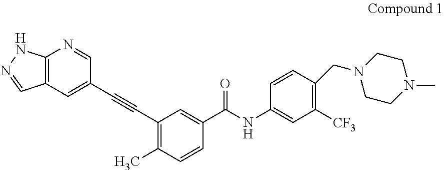 [SMILES: Cc1ccc(C(=O)Nc2ccc(CN3CCN(C)CC3)c(C(F)(F)F)c2)cc1C#Cc1cnc2[nH]ncc2c1]

or a pharmaceutically acceptable salt thereof.

Compounds of formula (I) and Compound 1 are small molecule inhibitors of Abelson tyrosine kinase (ABL). Among the drugs or drug candidates screened in the effort to repurpose existing compounds for the treatment of coronavirus infections, ABL inhibitors have shown promising activities against coronaviruses including SARS-CoV and MERS-CoV through a variety of mechanisms of action.

It has been reported that the first and second generations ABL inhibitors, Imatinib and Dasatinib, inhibit SARS-CoV and MERS-CoV with low cytotoxicity in Vero E6 cells. Dyall et al., Antimicrob Agents Chemother, 58 (8), 4885-93.

In another study on the effect of Imatinib against coronaviruses, Imatinib showed inhibition against virus production of SARS-CoV in Vero E6 and Calu-3 cells or MERS-CoV in Vero E6 and MRC5 cells. Coleman et al., Journal of Virology, 90 (19), 8924-33.

It has also been reported that Imatinib can inhibit viral membrane fusion. Coleman et al., Journal of Virology, 90 (19), 8924-33. Since virus replication requires entry of viruses into host cells by fusing with cellular membranes, blocking coronavirus spike protein induced fusion could inhibit virus replication and treat coronavirus infections.

Since the outbreak of COVID-19, it has been reported that a subgroup of patients with severe COVID-19 appear to have a cytokine storm syndrome (CSS), which is the main cause of death. Mehta et al., The Lancet, 395, 10229, 1033-1034. Cytokine storm is caused by excessive or uncontrolled levels of cytokines released by the immune system. It causes the activation of more immune cells and results in hyperinflammation. This can seriously harm or even kill the patient. Therapeutic options leading to immunosuppression including selective cytokine blockade are thus likely to be beneficial.

ABL kinase inhibitors, Nilotinib and Ponatinib, were reported to inhibit the production of multiple pro-inflammatory cytokines. Chen et al., Front. Immunol. 10: 1393. The study reports that Nilotinib and Ponatinib inhibit the production of three important cytokines, interleukin 8 (IL-8), interferon gamma-induced protein 10 (IP-10), and monocyte chemoattractant protein 1 (MCP-1), all of which are associated with influenza-induced cytokine storm.

As a third generation ABL kinase inhibitor, Ponatinib further showed to prolong the survival of mice with lethal influenza infection in animal models. Chen et al., Front. Immunol. 10: 1393. The suppression of the cytokine production and the prolonged survival further indicate the potential use of ABL inhibitors in the treatment of COVID-19 patients with severe symptom via suppressing cytokine storm.

In yet another study, Ponatinib showed inhibition against receptor-interacting serine/threonine-protein kinase 1 (RIPK1). Gordon et al., BioRxiv, 2020.03.22, 002386. In this study, a SARS-CoV-2-Human protein-protein interaction map has identified RIPK1 as an interactor of SARS-CoV-2 protein Nsp12. Because Ponatinib is an inhibitor of RIPK1, it is therefore proposed that Ponatinib could be repurposed as a potential antiviral drug for treating COVID-19.

Without wishing to be bound by the above mechanisms of action of the ABL inhibitors on coronaviruses and viral infections, the present disclosure provides methods of treating coronavirus infections with compounds of formula (I) or Compound 1, or a pharmaceutically acceptable salt thereof, which are potent ABL inhibitors, and are thus potential antiviral compounds for treating coronavirus infections.

The chemical name for Compound 1 is 3-(2-(1H-pyrazolo[3,4-b]pyridin-5-yl)ethynyl)-4-methyl-N-(4-((4-methylpiperazin-1-yl)methyl)-3-(trifluoromethyl)phenyl)-benzamide.

As used herein, the compounds of formula (I) and Compound 1 include any tautomer forms. As a non-limiting example, tautomerization may occur in the pyrazole and pyrimidine groups.

The compounds of formula (I) or Compound 1 or a pharmaceutically acceptable salt thereof can be obtained according to the production methods described in U.S. Pat. No. 8,846,671 B2, issued Sep. 30, 2014, which is incorporated herein by reference in its entirety and for all purposes, or a method analogous thereto.

In certain embodiments, the viral infection is caused by a coronavirus.

In certain embodiments, the coronavirus infection is caused by a coronavirus including SARS-CoV-2, SARS-CoV, MERS-Cov, 229E, NL63, OC43, and HKUl.

In certain embodiments, the coronavirus infection is caused by a coronavirus including SARS-CoV-2, SARS-CoV, and MERS-CoV.

In certain embodiments, the coronavirus infection is caused by SARS-CoV-2.

In certain embodiments, the patient is suffering from COVID-19.

In certain embodiments, the coronavirus infection is caused by SARS-CoV.

In certain embodiments, the coronavirus infection is caused by MERS-CoV.

In certain embodiments, the coronavirus replication is inhibited.

In certain embodiments, the coronavirus polymerase is inhibited.

In certain embodiments, the coronaviral membrane fusion is inhibited.

In certain embodiments, an ABL signaling pathway is inhibited.

In certain embodiments, an ABL2 specific signaling pathway is inhibited.

In certain embodiments, a receptor-interacting serine/threonine-protein kinase 1 (RIRK1) is inhibited.

In certain embodiments, the method is in the treatment of the patient with cytokine storm syndrome.

In certain embodiments, the patient's production of cytokines is inhibited.

In certain embodiments, the patient's production of IL-8, IP-10 and MCP-1 is inhibited.

In certain embodiments, the inhibition is in vitro or in vivo.

Similar to Ponatinib, Compound 1 of the present invention is a third generation ABL inhibitor, equally inhibiting both ABL1 and ABL2 kinase, and has a greater potency compared to other ABL inhibitors including Imatinib, Dasatinib, Nilotinib and Ponatinib. Table 1 shows the inhibition activity of Compound 1 against ABL kinases.

TABLE 1
Compound 1 kinase inhibition activity.

| | Compound 1 | | |
|---|---|---|---|
| | % Ctrl | % Ctrl | % Ctrl |
| Kinase | @ 10 nM | @ 100 nM | @ 1000 nM |
| ABL1- | 4.4 | 0.1 | 0 |
| nonphosphorylated | | | |
| ABL1-phosphorylated | 3 | 0 | 0 |
| ABL2 | 2.6 | 0 | 0 |

Table 2 below shows Compound 1 has greater potency against ABL kinase compared to other ABL inhibitors demonstrating Compound 1 may have a greater potential as an antiviral compound against coronaviruses including SARS-CoV-2.

TABLE 2
Compound 1 activities.

| | Kinase Inhibition | Cellular Activity (BCR- |
|---|---|---|
| IC₅₀ (nM) | (BCR-ABL1 kinase) | ABL1 expressing Ba/F3) |
| Imatinib | 210 ± 21 | 565 ± 656 |
| Dasatinib | 0.13 ± 0.0039 | 31 ± 4 |
| Nilotinib | 8.8 ± 0.45 | 10 ± 3 |
| Ponatinib | 0.33 ± 0.03 | 11 ± 3 |
| Compound 1 | 0.30 ± 0.0045 | 6 ± 3 |

In another aspect, the present disclosure relates to a method of treating a viral infection including a coronavirus infection in a human in need thereof, comprising administering to the patient a therapeutically effective amount of a compound of formula (II):

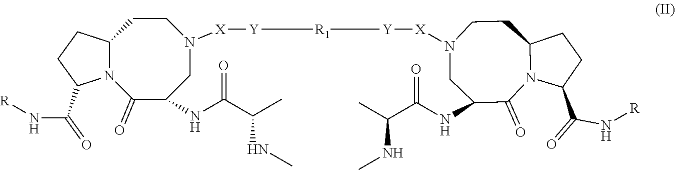

or a pharmaceutically acceptable salt thereof, wherein

X is selected from —C(═O—, —C(═S)—, —C(═NH)—, and —SO₂—;

Y is selected from —NH—, —O—, —S—, and absence;

R is selected from

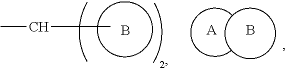

—C₃₋₆ cycloalkylene

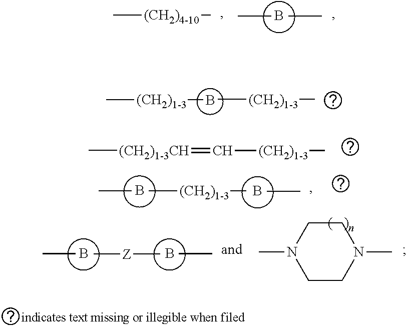

R₁ is selected from

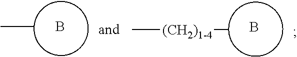

Z is O, S or NH;

n is 0, 1 or 2;

Ring A is a C₄₋₈ aliphatic ring; and

Ring B is phenyl, naphthyl, pyridyl, pyridazinyl, pyrazinyl or pyrimidinyl, and Ring B is optionally substituted.

In one embodiment of the compound of formula (II), X is C═O or —SO₂—.

In another embodiment of the compound of formula (II), X is —SO₂—.

In one embodiment of the compound of formula (II), Y is absent.

In another embodiment of the compound of formula (II), R is

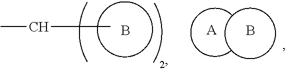

In another embodiment of the compound of formula (If), R is

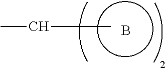

In another embodiment of the compound of formula (II), R₁ is selected from

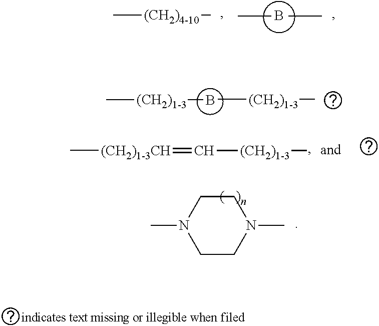

In another embodiment of the compound of formula (If), R₁ is

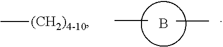

In another embodiment of the compound of formula (II), R₁ is

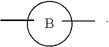

In another embodiment of the compound of formula (II), n is 0 or 1.

In another embodiment of the compound of formula (II), ring A is a C5-6 aliphatic ring.

In another embodiment of the compound of formula (II), ring B is phenyl, pyridyl, pyridazinyl, pyrazinyl or pyrimidinyl.

In another embodiment of the compound of formula (II), ring B is phenyl or pyridyl.

In another embodiment of the compound of formula (II), ring B is phenyl.

In another embodiment of the compound of formula (II), R is

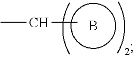

### R₁ is

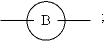

and ring B is phenyl.

In another embodiment of the compound of formula (II), X is C═O or —SO₂—; Y is absent; R is

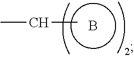

R₁ is

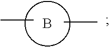

and ring B is phenyl or pyridyl.

In one embodiment, the compound of formula (II) is Compound 2:

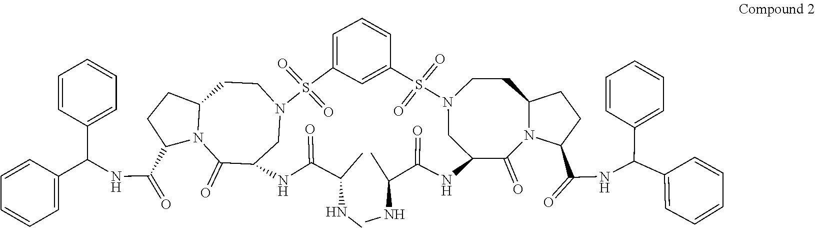

or a pharmaceutically acceptable salt thereof.

Compounds of formula (II) and Compound 2 are small molecule inhibitors of Inhibitors of Apoptosis Proteins (IAPs) proteins. Apoptosis, or programmed cell death, is a cell process critical for homeostasis, normal development, and host defense. Faulty regulation of apoptosis has been implicated in many human diseases, including viral infections. D. W. Nicholson, Nature 2000, 407, 810-816. Campbell G R et al., 2019, Autophagy, 15(4): 744-746. As a consequence, targeting of key apoptosis regulators has great potential for the development of new approaches to treat viral infections.

One class of regulators of apoptosis is the IAPs. This class includes proteins such as XIAP, cIAP1, cIAP2, ML-IAP, HIAP, KIAP, TSIAP, NAIP, survivin, livin, ILP-2, apollon, and BRUCE. IAP proteins potently suppress cell apoptosis. Among the IAPs, cellular IAP1 (cIAP1) and cIAP2 play a key role in the regulation of death-receptor mediated apoptosis, whereas X-linked IAP (XIAP) inhibits both death-receptor mediated and mitochondria mediated apoptosis by binding to and inhibiting caspase-3/7 and caspase-9, three cysteine proteases critical for execution of apoptosis. G. S. Salvesen et al., Nat. Rev. Mol. Cell. Biol. 2002, 3, 401-410.

Inhibitors of LAP are thus useful tools to elucidate the anti-apoptotic function of IAPs. IAP inhibitors have been reported to have promising activities against viral infections including HIV and Hepatitis B virus (HBV) infections.

In recent studies, IAP inhibition has been reported to be a new target for virus clearance. Campbell G R et al., 2019, Autophagy, 15(4): 744-746. The result showed that LAP inhibitor selectively kills HIV-1-infected resting memory CD4+ T cells, but not uninfected T_{CM}, resulting in the selective apoptosis of only the infected HIV-T_{CM}, while sparing uninfected bystander cells in the absence of viral infection.

IAP inhibitors have also been reported to selectively kill HBV-infected hepatocytes. In one study, HBV infection in two mouse models were induced to examine the relevance of host factors in controlling infection. Ebert G, et al. Proc Natl Acad Sci USA, 2015, 112(18): 5797-5802. The result showed cIAPs restrict the death of infected hepatocytes and allow HBV viral persistence. Animals with a liver-specific cIAP1 and total cIAP2 deficiency efficiently control HBV infection compared with wild type mice, indicating that antagonizing the function of cIAPs may promote the clearance of HBV infection.

In another study, inhibitors of cIAPs showed potential efficacy in treating HBV infection and other intracellular infections. Ebert G, et al. Proc Natl Acad Sci USA, 2015, 112(18): 5803-5808. In this study, an immunocompetent mouse model of chronic HBV infection was used to examine the in vivo efficacy of inhibitors of cIAPs in the treatment of HBV infection. The data shows Birinapant, a small molecule IPA inhibitor, was able to rapidly reduce serum HBV DNA and serum HBV surface antigen, and promote the elimination of hepatocytes containing HBV core antigen.

Without wishing to be bound by the above mechanisms of action of the TAP inhibitors, the present disclosure provides a method of treating coronavirus infections with compounds of formula (II) or Compound 2, or a pharmaceutically acceptable salt thereof, which are potent IAP inhibitors, and are thus potential antiviral compounds for treating coronavirus infections.

The chemical name for Compound 2 is (5S,5′S,8S,8′S,10aR,10a′R)-3,3′-(1,3-phenylenedisulfonyl)bis(N-benzhydryl-5-((S)-2-(methylamino)propanamido)-6-oxodecahydropyrrolo[1,2-a][1,5]diazocine-8-carboxamide).

As used herein, the compounds of formula (II) and Compound 2 include any tautomer forms. As a non-limiting example, tautomerization may occur in the pyrazole and pyrimidine groups.

The compound of formula (II) or Compound 2 or a pharmaceutically acceptable salt thereof can be obtained according to the production methods described in U.S. Pat. No. 8,883,771 B2, issued Nov. 11, 2014, which is incorporated herein by reference in its entirety and for all purposes, or a method analogous thereto.

In certain embodiments, the viral infection is caused by a coronavirus.

In certain embodiments, the coronavirus infection is caused by a coronavirus including SARS-CoV-2, SARS-CoV, MERS-CoV, 229E, NL63, OC43, and HKUl.

In certain embodiments, the coronavirus infection is caused by a coronavirus including SARS-CoV-2, SARS-CoV, and MERS-CoV.

In certain embodiments, the coronavirus infection is caused by SARS-CoV-2.

In certain embodiments, the patient is suffering from COVID-19.

In certain embodiments, the coronavirus infection is caused by SARS-CoV.

In certain embodiments, the coronavirus infection is caused by MERS-CoV.

In certain embodiments, the coronavirus replication is inhibited.

In certain embodiments, the coronavirus polymerase is inhibited.

In certain embodiments, IAP proteins are inhibited.

In certain embodiments, cIAP proteins are inhibited.

In certain embodiments, apoptosis in coronavirus infected cells is selectively induced.

In certain embodiments, coronavirus clearance is achieved.

In certain embodiments, coronavirus clearance is achieved by enhancing T cells mediated anti-virus immunity.

In certain embodiments, the inhibition is in vitro or in vivo.

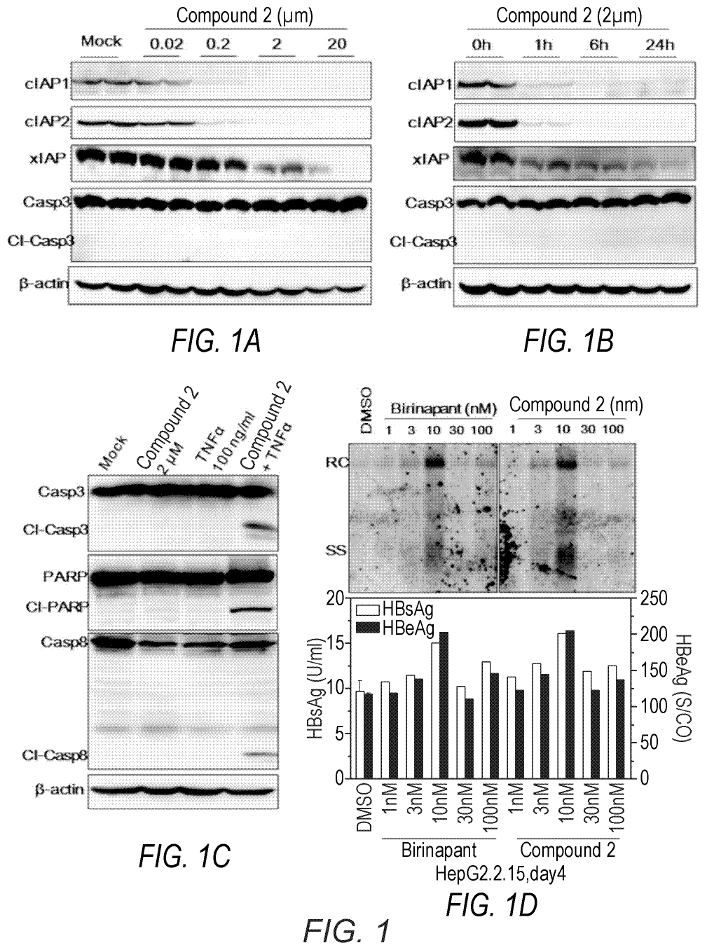

Similar to the above mentioned IAP inhibitors, Compound 2 of the present invention is a potent IAP inhibitor and can promote apoptosis, and thus could be a potential antiviral compound for coronavirus infection treatment. FIGS. 1A-1C show the effect of Compound 2 on apoptotic induction, IAPs expression, and HBV replication and gene expression in HepG2.2.15 liver cancer cell line.

As can be seen in FIG. 1, Compound 2 inhibits the expression of IAPs in a dose-dependent manner. The combination of Compound 2 and TNF-α significantly activate apoptosis pathway. As shown in FIG. 1D, low concentrations of Compound 2 and Birinapant increased virus replication by inhibiting cIAP2 expression, and high concentrations of Compound 2 and birinapant impair enhancement of virus replication by inhibiting cell proliferation through down-regulation of other IAP proteins.

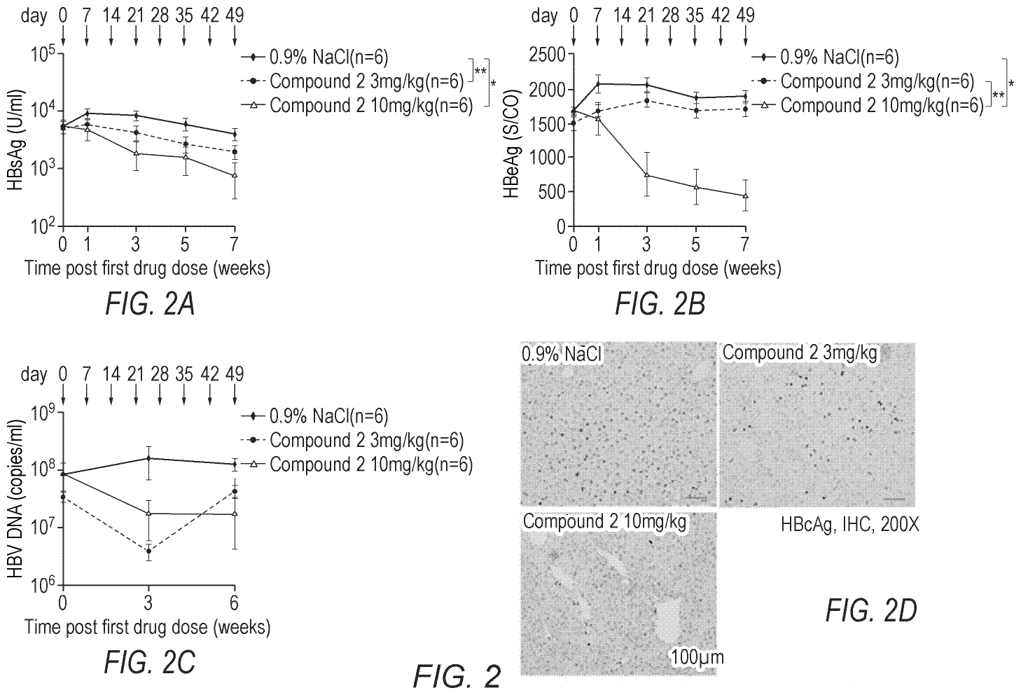

Injection of Compound 2 also promotes virus clearance in pAAV-HBV 1.3 model in C57BL/6J mice, as shown in FIGS. 2A-2D.

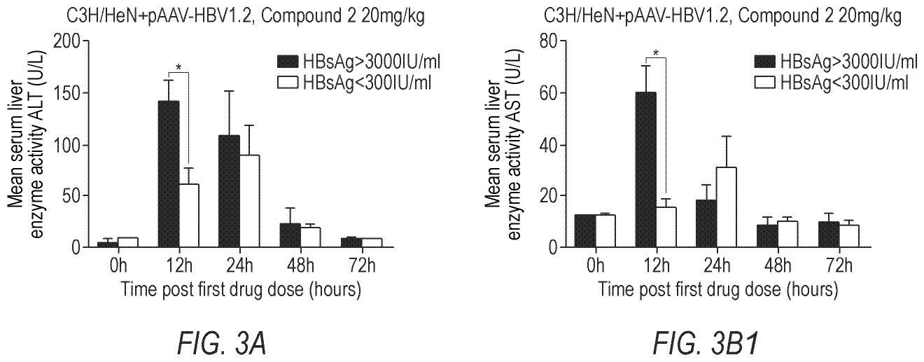

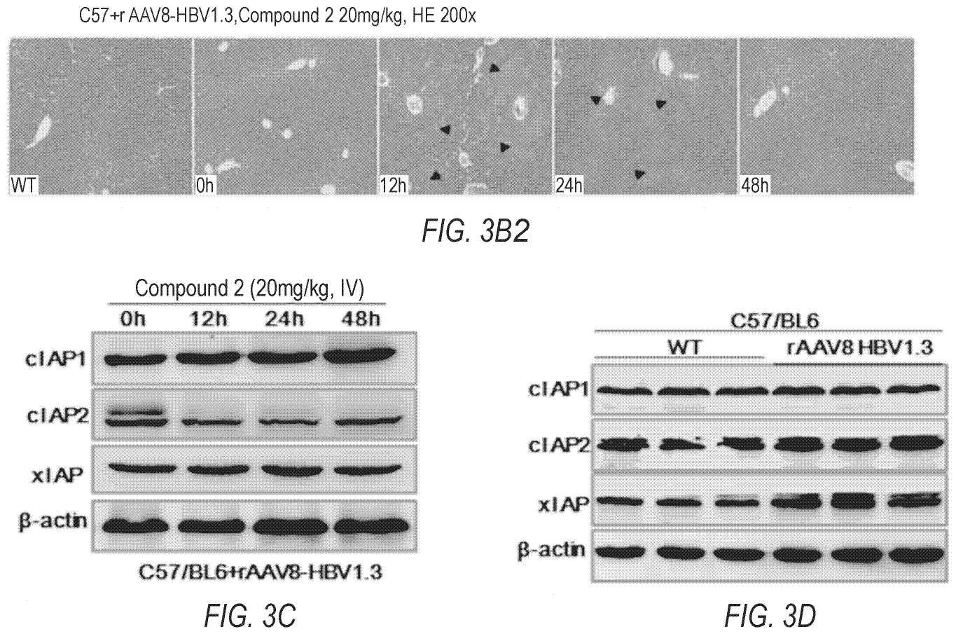

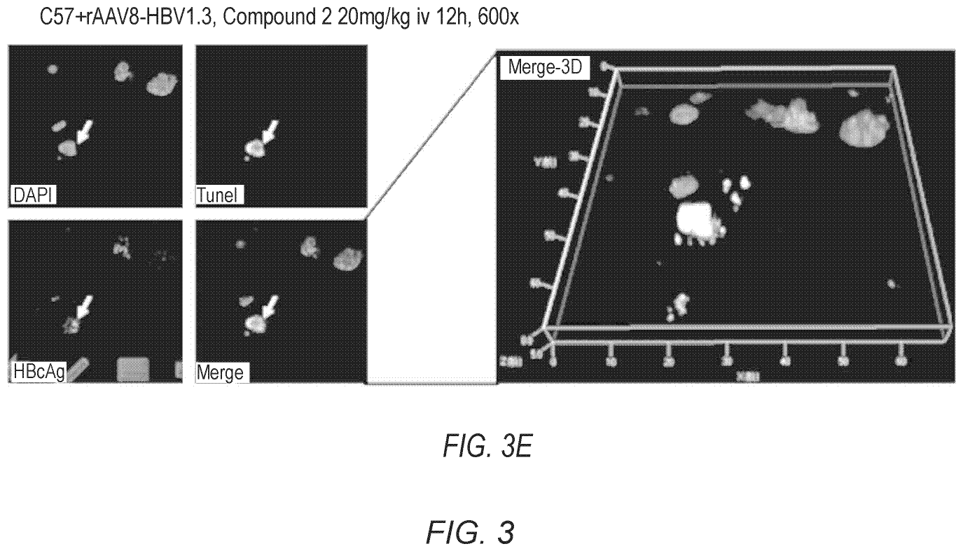

FIGS. 3A-3D shows that Compound 2 specifically induced apoptosis in the HBV-infected (HBsAg+) hepatocytes of HBV models in mice.

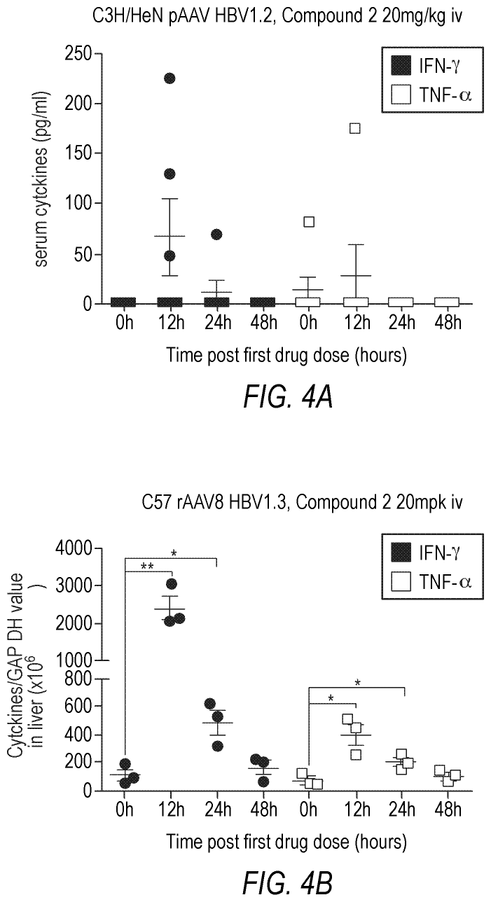

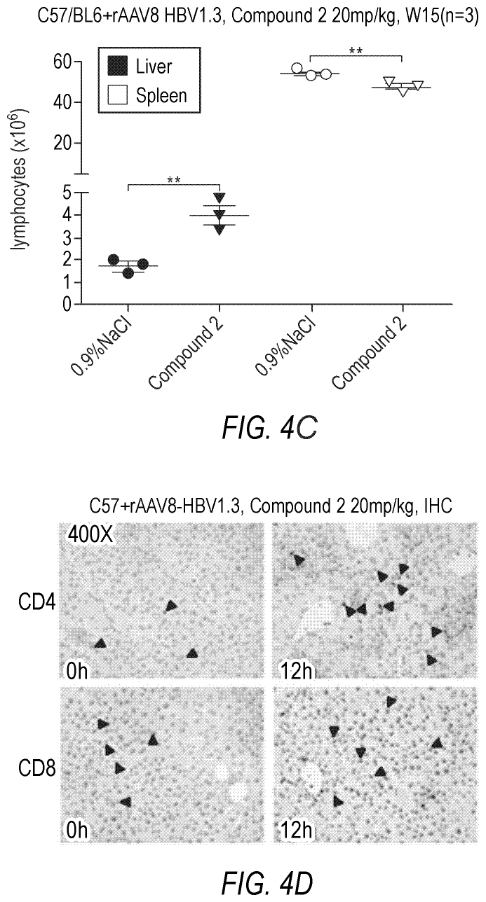

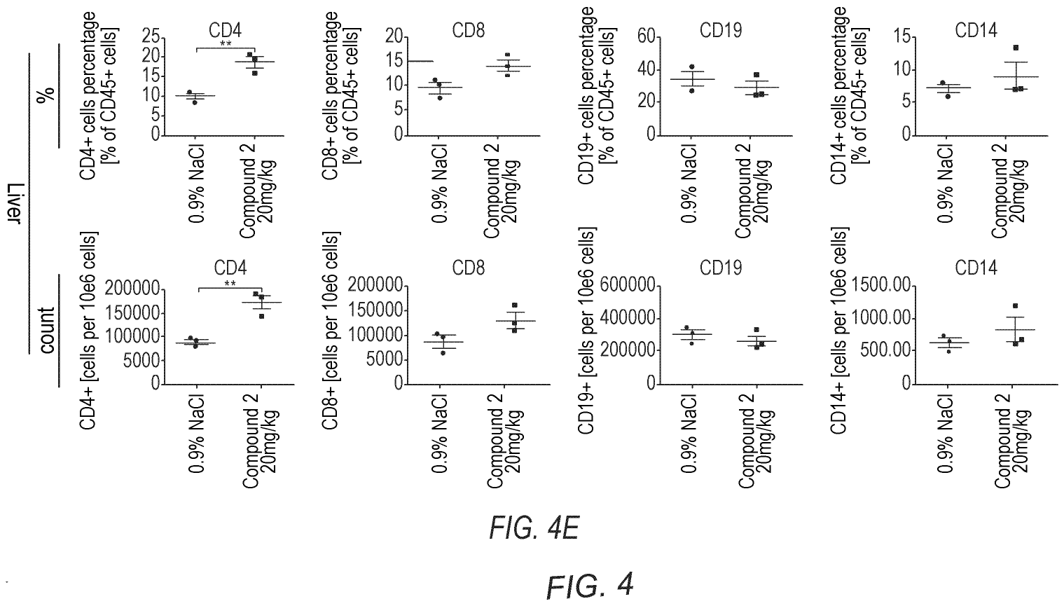

FIGS. 4A-4E show that Compound 2 promotes the expression of IFN-γ and TNF-α, and the number of CD4+ and CD8+ T cells in liver tissue of HBV models.

In another aspect, the present disclosure relates to a method of treating a viral infection including a coronavirus infection in a human in need thereof, comprising administering to the patient a therapeutically effective amount of a compound of formula (III), (IV), or (V):

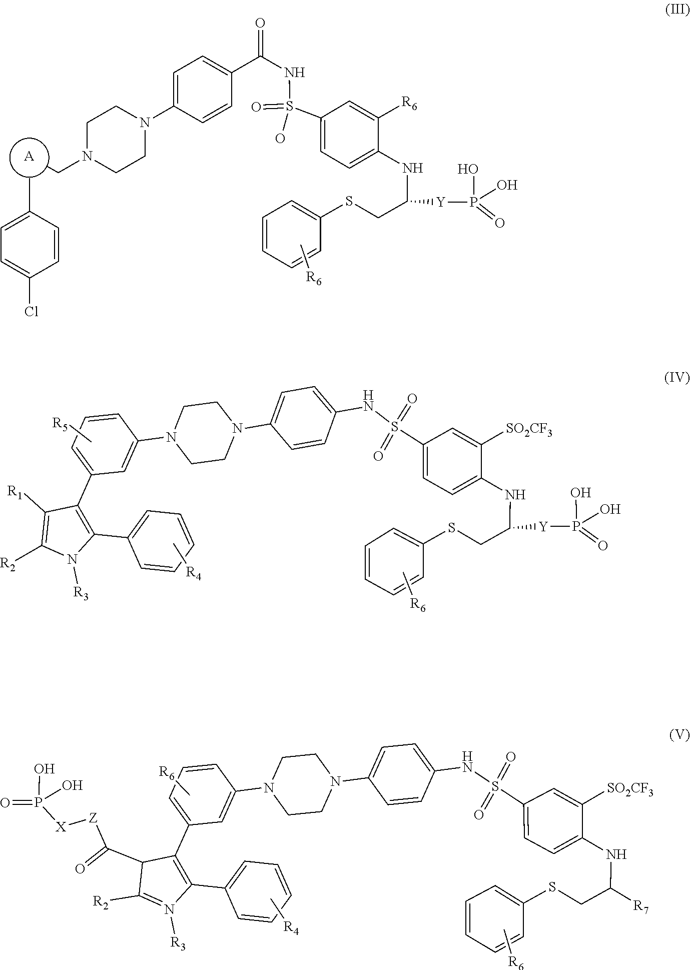

or a pharmaceutically acceptable salt thereof, wherein

wherein the A ring is

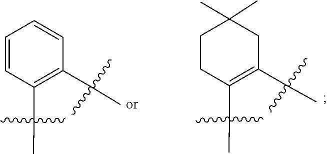

X, substituted or unsubstituted, is selected from the group consisting of alkylene, alkenylene, cycloalkylene, cycloalkenylene, and heterocycloalkylene;

Y is selected from the group consisting of (CH₂)ₙ—N(R^{a})₂ and

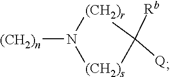

Q is selected from the group consisting of O, O(CH₂)₁₋₃, NR^{c}, NR^{c}(C₁₋₃ alkylene), OC(═O)(C₁₋₃ alkylene), C(═O)O, C(═O)O(C₁₋₃ alkylene), NHC(═O)(C₁₋₃ alkylene), C(═O)NH, and C(═O)NH(C₁₋₃ alkylene);

Z is O or NR^{c};

R₁ and R₂, independently, are selected from the group consisting of H, CN, NO₂, halo, alkyl, cycloalkyl, alkenyl, cycloalkenyl, alkynyl, aryl, heteroaryl, heterocycloalkyl, OR′, SR′, NR′R″, COR′, CO₂R′, OCOR′, CONR′R″, CONR′SO₂R″, NR′COR″, NR′CONR″R′″, NR′C═SNR″R′″, NR′SO₂R″, SO₂R′, and SO₂NR′R″;

R₃ is selected from a group consisting of H, alkyl, cycloalkyl, alkenyl, cycloalkenyl, alkynyl, aryl, heteroaryl, heterocycloalkyl, OR′, NR′R″, OCOR′, CO₂R′, COR′, CONR′R″, CONR′SO₂R″, C₁₋₃ alkyleneCH(OH)CH₂OH, SO₂R′, and SO₂NR′R″;

R′, R″, and R′″, independently, are H, alkyl, cycloalkyl, alkenyl, cycloalkenyl, alkynyl, aryl, heteroaryl, C₁₋₃ alkyleneheterocycloalkyl, or heterocycloalkyl;

R′ and R″, or R″ and R′″, can be taken together with the atom to which they are bound to form a 3 to 7 membered ring;

R₄ is hydrogen, halo, C₁₋₃ alkyl, CF₃, or CN;

R₅ is hydrogen, halo, C₁₋₃ alkyl, substituted C₁₋₃ alkyl, hydroxyalkyl, alkoxy, or substituted alkoxy;

R₆ is selected from the group consisting of H, CN, NO₂, halo, alkyl, cycloalkyl, alkenyl, cycloalkenyl, alkynyl, aryl, heteroaryl, heterocycloalkyl, OR′, SR′, NR′R″, CO₂R′, OCOR′, CONR′R′″, CONR′SO₂R″, NR′COR″, NR′CONR″R′″, NR′C═SNR″R′″, NR′SO₂R″, SO₂R′, and SO₂NR′R″;

R₇, substituted or unsubstituted, is selected form the group consisting of hydrogen, alkyl, alkenyl, (CH₂)₀₋₃ cycloalkyl, (CH₂)₀₋₃ cycloalkenyl, (CH₂)₀₋₃ heterocycloalkyl, (CH₂)₀₋₃ aryl, and (CH₂)₀₋₃ heteroaryl;

R₈ is selected form the group consisting of hydrogen, halo, NO₂, CN, CF₃SO₂, and CF₃;

Rₐ is selected from the group consisting of hydrogen, alkyl, heteroalkyl, alkenyl, hydroxyalkyl, alkoxy, substituted alkoxy, cycloalkyl, cycloalkenyl, and heterocycloalkyl;

R_{b} is hydrogen or alkyl;

R_{c} is selected from the group consisting of hydrogen, alkyl, substituted alkyl, hydroxyalkyl, alkoxy, and substituted alkoxy; and

n, r, and s, independently, are 1, 2, 3, 4, 5, or 6;

In one embodiment of the compound of formula (III), (IV), or (V), ring A is

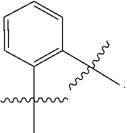 [SMILES: *c1ccccc1*]

In another embodiment of the compound of formula (III), (IV), or (V), X is alkylene. In another embodiment, X is C₁₋₄ alkylene.

In one embodiment of the compound of formula (III), (IV), or (V), Y is

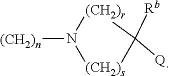

In another embodiment of the compound of formula (III), (IV), or (V), Q is C(═O)O(C₁₋₃ alkylene).

In another embodiment of the compound of formula (III), (IV), or (V), Z is O.

In another embodiment of the compound of formula (III), (IV), or (V), R₁ is SO₂R′, and R′ is alkyl. In another embodiment, R₁ is SO₂Me.

In another embodiment of the compound of formula (III), (IV), or (V), R₂ is alkyl. In another embodiment, SO₂ is methyl, propyl, or isopropyl. In another embodiment, SO₂ is methyl.

In another embodiment of the compound of formula (III), (IV), or (V), R₃ is H or alkyl. In another embodiment, R₃ is C₁₋₄ alkyl. In another embodiment, R₃ is methyl, propyl, or isopropyl. In another embodiment, R₃ is isopropyl.

In another embodiment of the compound of formula (III), (IV), or (V), R₄ is hydrogen or halo. In another embodiment, R₄ is halo. In another embodiment, R₄ is —Cl or —F. In another embodiment, R₄ is —Cl.

In another embodiment of the compound of formula (III), (IV), or (V), R₅ is hydrogen or halo. In another embodiment, R₅ is halo. In another embodiment, R₅ is —Cl or —F. In another embodiment, R₅ is —F.

In another embodiment of the compound of formula (III), (IV), or (V), R₆ is H or alkyl. In another embodiment, R₆ is H.

In another embodiment of the compound of formula (III), (IV), or (V), R₇ is H or alkyl. In another embodiment, R₇ is H.

In another embodiment of the compound of formula (III), (IV), or (V), R₈ is hydrogen, halo, or CF₃SO₂. In another embodiment, R₈ is CF₃SO₂.

In another embodiment of the compound of formula (III), (IV), or (V), n, r, and s are each independently 1 or 2. In another embodiment, n, r, and s are 2. In another embodiment, n is 1; and r and s are 2.

In another embodiment of the compound of formula (III), (IV), or (V), R^{b} is H.

In one embodiment, the compound of formula (III) or (IV) is Compound 3:

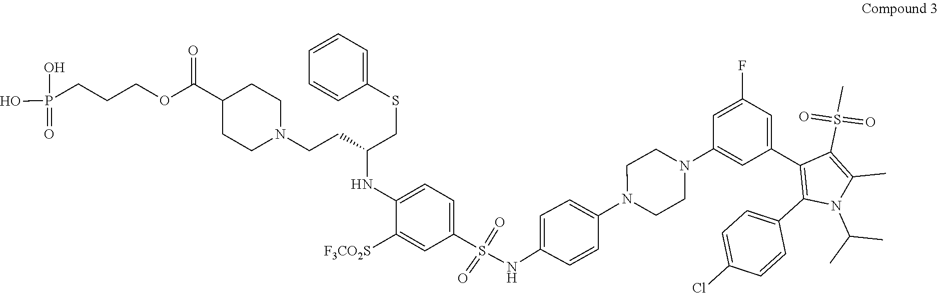

or a pharmaceutically acceptable salt thereof.

In another embodiment, the present invention relates to a method for treating a viral infection in a patient, comprising administering to the patient a therapeutically effective amount of Compound 3.

In another aspect, the present disclosure relates to a method of treating a viral infection including a coronavirus infection in a human in need thereof, comprising administering to the patient a therapeutically effective amount of a compound of formula (VI):

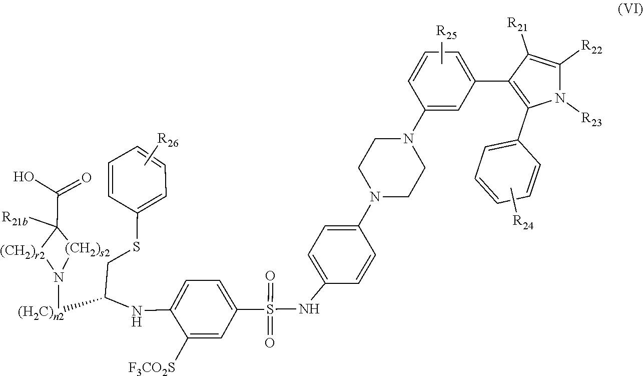

or a pharmaceutically acceptable salt thereof wherein

SO₂₁ is SO₂SO₂′,

SO₂₂ is alkyl, preferably C₁-C₄ alkyl, more preferably methyl, propyl, or isopropyl,

SO₂₃ is alkyl, preferably C₁-C₄ alkyl, more preferably methyl, propyl, or isopropyl,

SO₂₄ is halogen, preferably fluoride, chloride,

SO₂₅ is halogen, preferably fluoride, chloride,

SO₂₆ is selected from H, halogen, alkyl, preferably fluoride, chloride, C₁-C₄ alkyl, more preferably methyl, propyl, isopropyl

SO_{21b} is H or alkyl, preferably C₁-C₄ alkyl, more preferably methyl, propyl, or isopropyl,

n₂, SO₂ and s₂ are independently 1, 2, 3, 4, 5 or 6, more preferably, SO₂ and s₂ are both 2 and n₂ is 3, 4 or 5, more preferably, all of n₂, SO₂ and s₂ are 2, and

SO₂′ is alkyl, preferably C₁-C₄ alkyl, more preferably methyl, propyl, or isopropyl.

In one embodiment of the compound of formula (VI), SO₂₁ is SO₂(C₁-C₄ alkyl). In another embodiment, SO₂₁ is SO₂Me.

In another embodiment of the compound of formula (VI), SO₂₂ is C₁-C₄ alkyl. In another embodiment, SO₂₂ is methyl.

In another embodiment of the compound of formula (VI), SO₂₃ is C₁₋₄ alkyl. In another embodiment, SO₂₃ is isopropyl.

In another embodiment of the compound of formula (VI), SO₂₄ is fluoride or chloride. In another embodiment, SO₂₄ is chloride.

In another embodiment of the compound of formula (VI), SO₂₅ is fluoride or chloride. In another embodiment, SO₂₅ is fluoride.

In another embodiment of the compound of formula (VI), SO₂₆ is H or C₁-C₄ alkyl.

In another embodiment, SO₂₆ is H.

In another embodiment of the compound of formula (VI), SO_{21b} is H.

In another embodiment of the compound of formula (VI), n₂, SO₂ and s₂ are each independently 1 or 2. In another embodiment, n₂, SO₂ and s₂ are 2.

In another embodiment of the compound of formula (VI), SO₂₁ is SO₂(C₁-C₄ alkyl); SO₂₂ is C₁-C₄ alkyl; and SO₂₃ is C₁-C₄ alkyl. In another embodiment, SO₂₁ is SO₂Me; SO₂₂ is methyl; and SO₂₃ is isopropyl.

In another embodiment of the compound of formula (VI), SO₂₄ is —Cl, and SO₂₅ is —F.

In one embodiment, the compound of formula (VI) includes Compound 4:

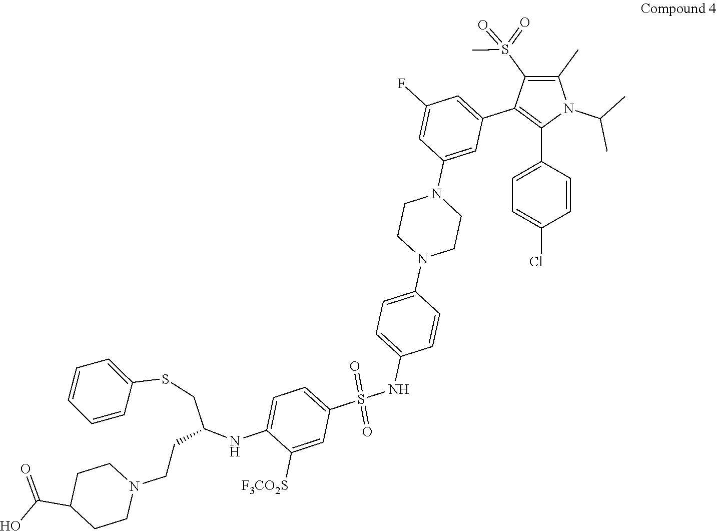

or a pharmaceutically acceptable salt thereof.

In another embodiment, the present invention relates to a method for treating a viral infection in a patient, comprising administering to the patient a therapeutically effective amount of Compound 4.

In another aspect, the present invention relates to a method for treating a viral infection in a patient, comprising administering to the patient a therapeutically effective amount of Compound 5:

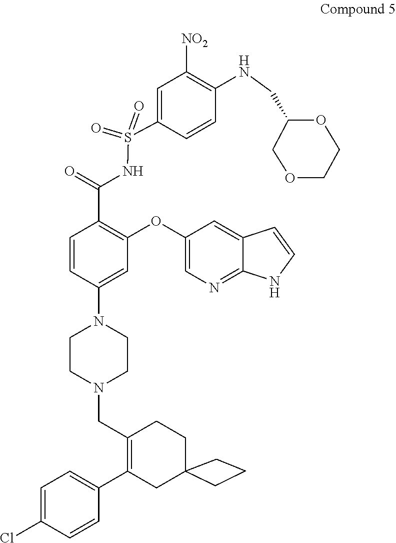 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

Compounds of formula (III), (IV), (V), or (VI), Compound 3, Compound 4, and Compound 5 are inhibitors of BCL-2 proteins. BCL-2 proteins function as critical regulators of apoptosis in cells. D. T. Chao, et al., Annu Rev Immunol 1998; 16:395-419. Bcl-2 proteins serve as a check on apoptosis allowing healthy and useful cells to survive. This protein family includes anti-apoptotic proteins, such as BCL-2, BCL-xL, and MCL-1, and pro-apoptotic molecules, including Bid, Bim, Bad, Bak and Bax. Replication of several important human microbes including coronaviruses, influenza A virus (IAV), HBV, HCV and HIV depend on BCL-2-, BCL-xL-, and BCL-w-mediated mitochondria-initiated apoptosis. Geng et al., Proc Natil Acad Sci USA 2012; 109: 18471-18476. Park J et al., Hepatology 2012; 56: 831-840. Busca A et al., J Biol Chem 2012; 287: 15118-15133. Tan et al., J Virol. 2007, 81: 6346-6355.

BCL-xL inhibitors, such as ABT-737 and its orally available analogue ABT-236, have been reported to accelerate apoptosis in virus-infected cells. Therefore, BCL-xL inhibitors could be potential antiviral compounds for treating viral infections.

In another study, it was found that ABT-263 accelerates apoptosis in virus-infected cells by activating the caspase-mediate apoptosis pathway, thus triggering deaths of influenza A virus (IAV) infected cells. Kakkola et al., Cell Death and Disease, 2013, 4, e742.

Without wishing to be bound by the above mechanisms of action of the BCL-xL inhibitors, the present disclosure provides a method of treating coronavirus infections with compounds of formula (III), (IV), (V), or (VI), or Compound 3, Compound 4, or Compound 5, or a pharmaceutically acceptable salt thereof, which are potent BCL-xL inhibitors, and are thus potential antiviral compounds for treating coronavirus infections.

The chemical name for Compound 3 is (R)-2-(1-(3-(4-(N-(4-(4-(3-(2-(4-chlorophenyl)-1-isopropyl-5-methyl-4-(methylsulfonyl)-1H-pyrrol-3-yl)-5-fluorophenyl)piperazin-1-yl)phenyl)sulfamoyl)-2-(trifluoromethylsulfonyl)phenylamino)-4-(phenylthio)butyl)piperidine-4-carbonyloxy)ethylphosphonic acid.

The chemical name for Compound 4 is (R)-1-(3-(4-(N-(4-(4-(3-(2-(4-chlorophenyl)-1-isopropyl-5-methyl-4-(methylsulfonyl)-1H-pyrrol-3-yl)-5-fluorophenyl) piperazin-1-yl)phenyl) sulfamoyl)-2-(trifluoromethylsulfonyl)phenylamino)-4-(phenylthio)butyl)piperidine-4-carboxylic acid.

The chemical name for Compound 5 is (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide.

As used herein, the compounds of formula (III), (IV), (V), or (VI), and Compounds 3, 4 and 5, include any tautomer forms. As a non-limiting example, tautomerization may occur in the pyrazole and pyrimidine groups.

The compound of formula (III), (IV), or (V), and Compounds 3 and 4 or a pharmaceutically acceptable salt thereof can be obtained according to the production methods described in U.S. Pat. No. 9,096,625 B2, issued Aug. 4, 2015, which is incorporated herein by reference in its entirety and for all purposes, or a method analogous thereto.

Compound 5 or a pharmaceutically acceptable salt thereof can be obtained according to the production methods described in U.S. Pat. No. 10,221,174 B, issued Mar. 5, 2019, which is incorporated herein by reference in its entirety and for all purposes, or a method analogous thereto.

In certain embodiments, the viral infection is caused by a coronavirus.

In certain embodiments, the coronavirus infection is caused by a coronavirus including SARS-CoV-2, SARS-CoV, MERS-CoV, 229E, NL63, OC43, and HKUl.

In certain embodiments, the coronavirus infection is caused by a coronavirus including SARS-CoV-2, SARS-CoV, and MERS-CoV.

In certain embodiments, the coronavirus infection is caused by SARS-CoV-2.

In certain embodiments, the patient is suffering from COVID-19.

In certain embodiments, the coronavirus infection is caused by SARS-CoV.

In certain embodiments, the coronavirus infection is caused by MERS-CoV.

In certain embodiments, the coronavirus replication is inhibited.

In certain embodiments, the coronavirus polymerase is inhibited.

In certain embodiments, BCL-2 proteins are inhibited.

In certain embodiments, BCL-xL proteins are inhibited.

In certain embodiments, the apoptosis in coronavirus infected cells is selectively induced.

In certain embodiments, the caspase-mediate apoptosis pathway is activated.

In certain embodiments, the inhibition is in vitro or in vivo.

Also provided herein are pharmaceutical compositions and dosage forms, comprising compounds of formula (I)-(VI) or Compounds 1-5 or a pharmaceutically acceptable salt thereof, and one or more pharmaceutically acceptable excipients.

Compositions and dosage forms provided herein may further comprise one or more additional active ingredients. Compounds of formula (I)-(VI) or Compounds 1-5 or a pharmaceutically acceptable salt thereof may be administered as part of a pharmaceutical composition as described.

A compound of the present disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a compound of the present disclosure is administered in an effective amount to achieve its intended purpose. The exact formulation, route of administration, and dosage is determined by an individual physician in view of the diagnosed condition or disease. Dosage amount and interval can be adjusted individually to provide levels of a compound of the present disclosure that is sufficient to maintain therapeutic effects.

Toxicity and therapeutic efficacy of the compounds of the present disclosure can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a compound of the present disclosure required for use in therapy varies with the nature of the condition being treated, the length of time that activity is desired, and the age and the condition of the subject, and is ultimately determined by the attendant physician. Dosage amounts and intervals can be adjusted individually to provide plasma levels of the compound of the present disclosure that are sufficient to maintain the desired therapeutic effects. The desired dose can be administered in a single dose, or as multiple doses administered at appropriate intervals, for example as one, two, three, four or more subdoses per day. Multiple doses often are desired, or required. For example, a compound of the present disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two-day rest, and another five daily doses (5/2/5); or, any dose regimen determined to be appropriate for the circumstance.

In some embodiments, compounds of the present disclosure typically are administered in admixture with a pharmaceutical carrier to give a pharmaceutical composition selected with regard to the intended route of administration and standard pharmaceutical practice.

Pharmaceutical compositions for use in accordance with the present disclosure are formulated in a conventional manner using one or more physiologically acceptable carriers comprising excipients and/or auxiliaries that facilitate processing of compound of the present disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the compound of the present disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a compound of the present disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a compound of the present disclosure.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay, and i) lubricants such as tale, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition such that they release the active ingredient(s) only, or preferentially, in a certain part of the intestinal tract, optionally, in a delayed manner. Examples of embedding compositions that can be used include polymeric substances and waxes.

In solid dosage forms the active ingredients may be mixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

The active ingredients can also be in micro-encapsulated form with one or more excipients as noted above.

When a therapeutically effective amount of a compound of the present disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the present disclosure can be readily combined with pharmaceutically acceptable carriers well-known in the art. Standard pharmaceutical carriers are described in Remington's Pharmaceutical Sciences, Mack Publishing Co., Easton, Pa., 19th ed. 1995. Such carriers enable the active agents to be formulated as tablets, pills, dragees, capsules, liquids, gels, syrups, slurries, suspensions and the like, for oral ingestion by a subject to be treated.

Pharmaceutical preparations for oral use can be obtained by adding the compound of the present disclosure to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Compounds of the present disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a compound of the present disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the present disclosure also can be formulated in rectal compositions, such as suppositories or retention enemas, e.g., containing conventional suppository bases. In addition to the formulations described previously, the compound of the present disclosure also can be formulated as a depot preparation. Such long-acting formulations can be administered by implantation (for example, subcutaneously or intramuscularly) or by intramuscular injection. Thus, for example, the compound of the present disclosure can be formulated with suitable polymeric or hydrophobic materials (for example, as an emulsion in an acceptable oil) or ion exchange resins.

In particular, the compounds of the present disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the present disclosure also can be injected parenterally, for example, intravenously, intramuscularly, subcutaneously, or intracoronarily.

For parenteral administration, the compound of the present disclosure are typically used in the form of a sterile aqueous solution which can contain other substances, for example, salts or monosaccharides, such as mannitol or glucose, to make the solution isotonic with blood.

In certain embodiments, the compound of formula (I)-(VI) or Compounds 1-5, or pharmaceutically acceptable salt thereof is administered orally to the patients in need such treatment.

In certain embodiments, the compound of formula (I)-(VI) or Compounds 1-5, or pharmaceutically acceptable salt thereof is administered intravenously to the patients in need such treatment.

A compound of the present disclosure used in a method of the present disclosure can be administered in an amount of about 0.005 to about 500 milligrams per dose, about 0.05 to about 250 milligrams per dose, or about 0.5 to about 100 milligrams per dose. For example, a compound of the present disclosure can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams.

The dosage of a composition containing a compound of the present disclosure, or a composition containing the same, can be from about 1 μg/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more.

In certain embodiments, the compound of formula (I)-(VI) or Compounds 1-5, or pharmaceutically acceptable salt thereof is administered every day, once a day, twice a day, three times a day, once every other day (QOD), or once every three days. The amount of administration is from 0.5 mg to 100 mg, preferably from 1 mg to 80 mg, more preferably from 1 mg to 60 mg. In the most preferable embodiments, it is in an amount of about 1 mg, 2 mg, 4 mg, 6 mg, 8 mg, 10 mg, 12 mg, 14 mg, 16 mg, 18 mg, 20 mg, 22 mg, 24 mg, 26 mg, 28 mg, 30 mg, 32 mg, 34 mg, 36 mg, 38 mg, 40 mg, 42 mg, 44 mg, 46 mg, 48 mg, 50 mg, 52 mg, 54 mg, 56 mg, 58 mg, or 60 mg.

In certain embodiments, the compound of formula (I)-(VI) or Compounds 1-5 is in a solid dosage form.

In certain embodiments, the present invention relates to use of a compound of formula (I)-(VI) or Compounds 1-5, or pharmaceutically acceptable salt thereof in the manufacture of medicament for the treatment of coronavirus infection.

The present disclosure describes various embodiments. A person of ordinary skill in the art reviewing the disclosure will readily recognize that various embodiments can be combined in any variation. For example, embodiments of the disclosure include treatment of various disorders, patient populations, administrations of dosage forms, at various dosages, minimization of various adverse events, and improvements in various efficacy measures, etc. Any combinations of various embodiments are within the scope of the disclosure.

### EXAMPLES

### Example 1 Dose Response Curve (DRC) Analysis by Immunofluorescence

Ten-point DRCs were generated for each drug. Vero cells were seeded at 1.2×10⁴ cells per well in DMEM, supplemented with 2% FBS and 1× antibiotic-antimycotic solution (Gibco), in black, 384-well μClear plates (Greiner Bio-One) 24 h prior to the experiment. Ten-point DRCs were generated with compound concentrations ranging from 0.1 to 50 μM. For the viral infections, plates were transferred into the BSL3 containment facility and SARS-CoV-2 was added at a multiplicity of infection (MOI) of 0.0125. The cells were fixed at 24 hours postinfection (hpi) with 4% PFA and analyzed by immunofluorescence. The acquired images were analyzed using in-house software to quantify cell numbers and infection ratios, and antiviral activity was normalized to positive (mock) and negative (0.5% DMSO) controls in each assay plate. DRCs were fitted by sigmoidal dose-response models, with the following equation: Y=bottom+(top−bottom)/[1+(IC₅₀/X)^{Hillslope}], using XLfit 4 software or Prism7.

IC₅₀ values were calculated from the normalized activity data set-fitted curves. All IC₅₀ and 50% cytotoxic concentration (CC₅₀) values were measured in duplicate, and the quality of each assay was controlled by Z′-factor and the coefficient of variation in percent (% CV).

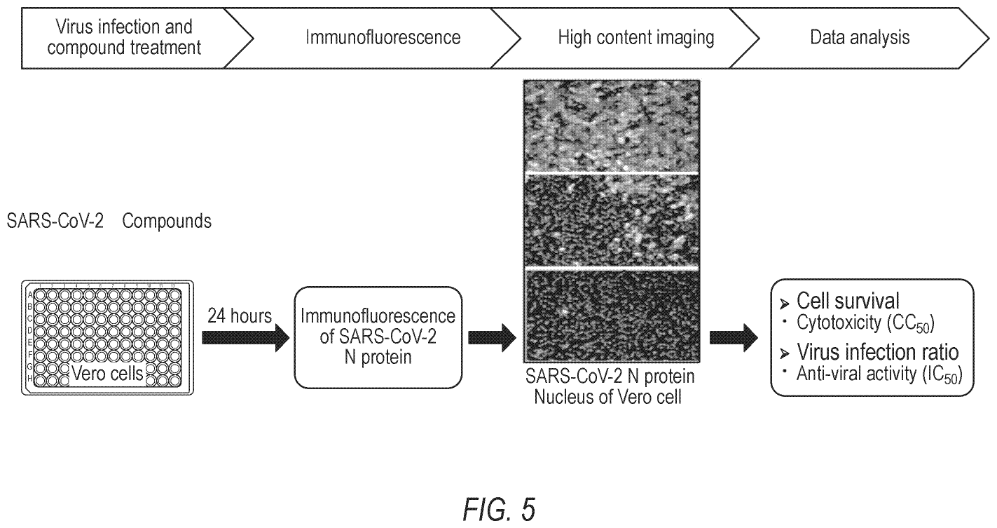

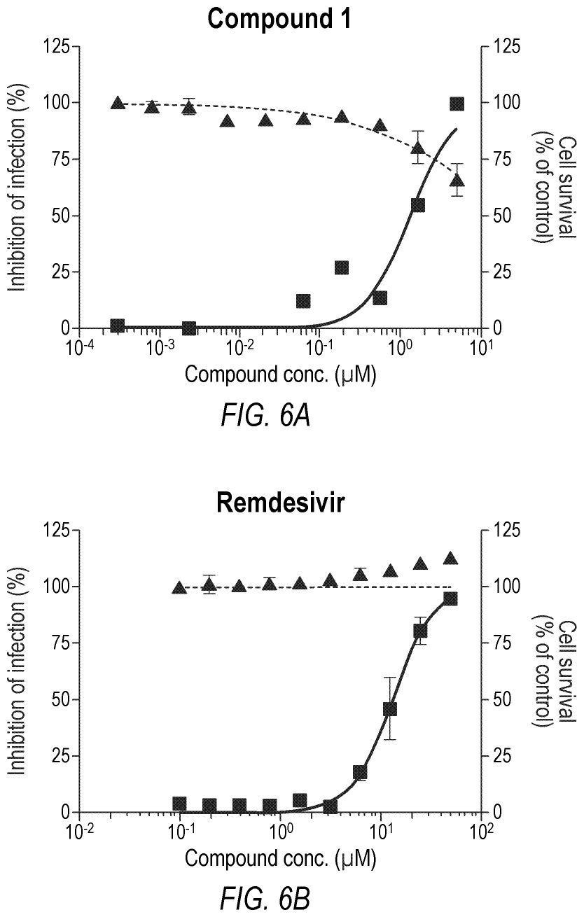

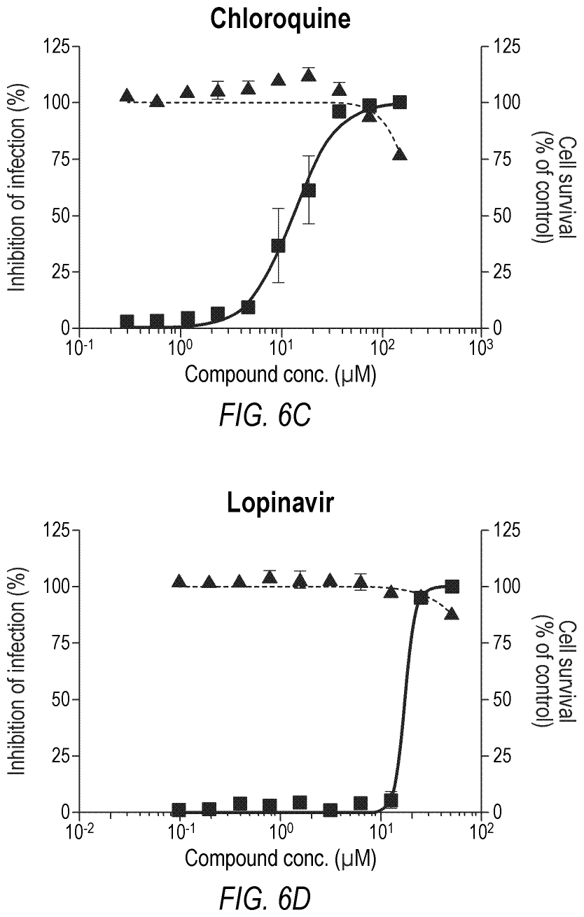

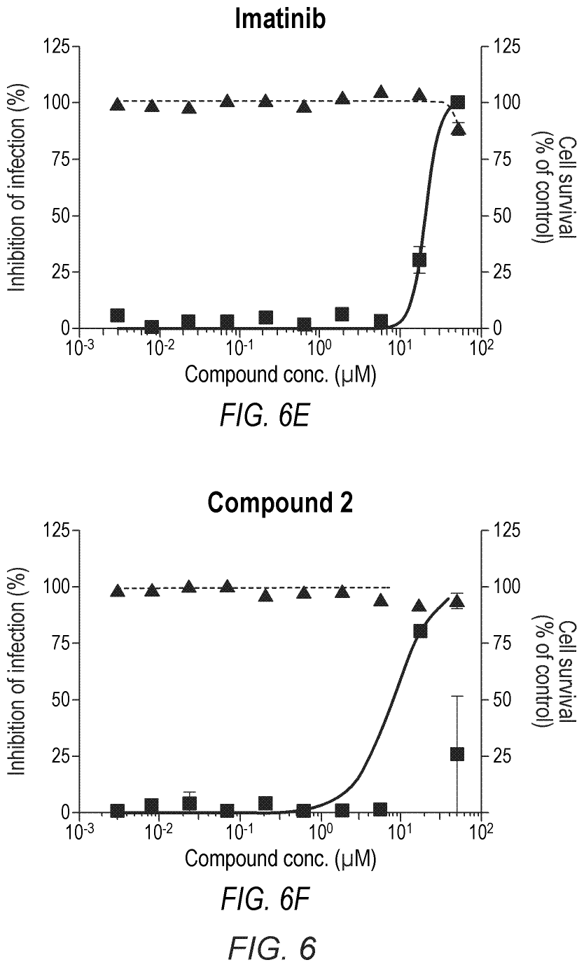

Following the procedure described above, Compound 1 and Compound 2 were tested in SARS-CoV-2 immunofluorescence assay (IFA) against Imatinib using Remdesivir, Chloroquine, and Lopinavir as references. The procedure and results are shown in Table 3, FIG. 5, and FIGS. 6A-6F.

TABLE 3

| | | Max | Max | | | |
|---|---|---|---|---|---|---|
| | | Conc. | Inhibition | IC₅₀ | CC₅₀ | |
| Compound | | (uM) | (%) | (uM) | (uM) | SI₅₀ |
| Test | Compound 1 | 5 | 99.71 | 1.32 | >5 | >3.8 |
| | Imatinib | 50 | 99.50 | 19.59 | >50 | >2.6 |
| | Compound 2 | 50 | 25.93 | >50 | >50 | NA |
| Reference | Remdesivir | 50 | 95.01 | 12.36 | >50 | >4.0 |
| | Chloroquine | 150 | 100.01 | 13.00 | >150 | >11.5 |
| | Lopinavir | 50 | 99.93 | 17.29 | >50 | >2.9 |

### Example 2 Inhibition of Cytokines Associated with Influenza Virus Induced Cytokine Storm

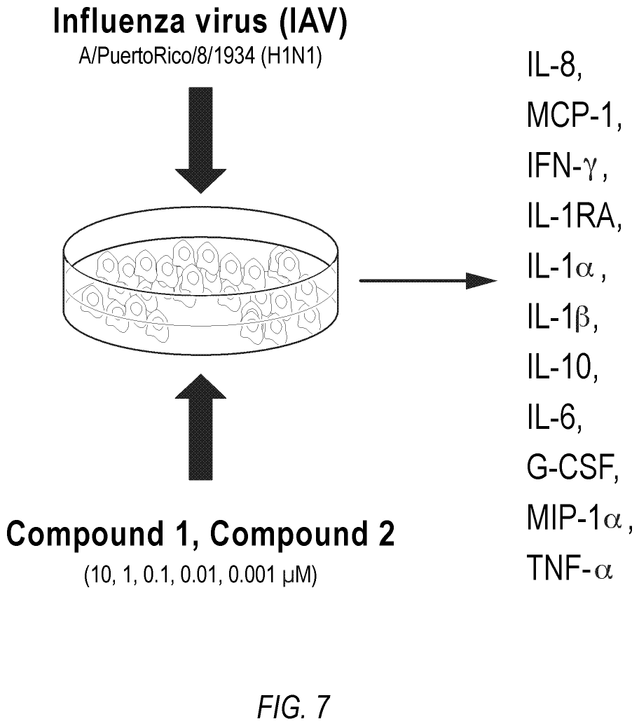

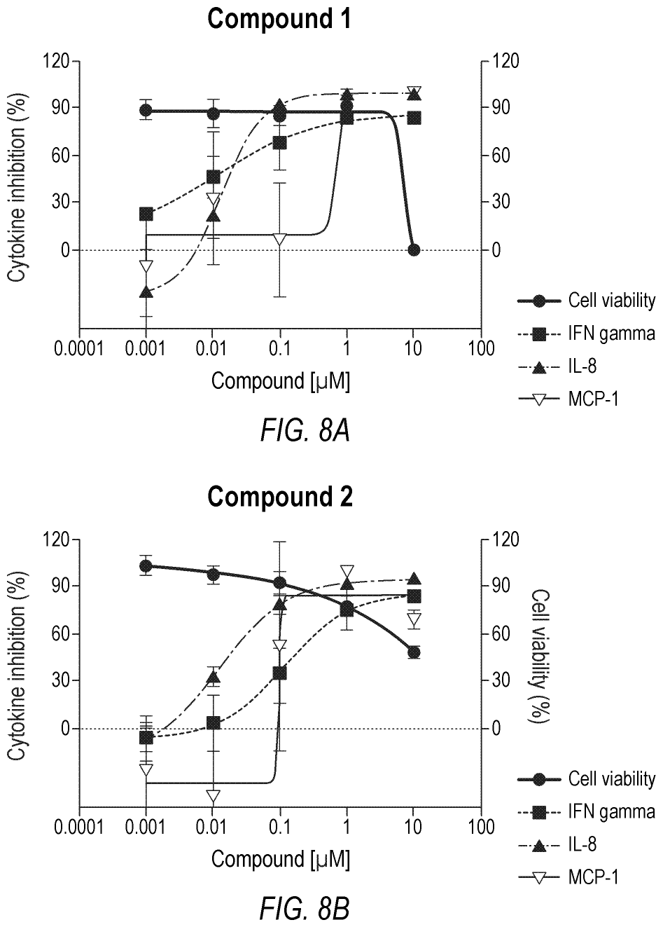

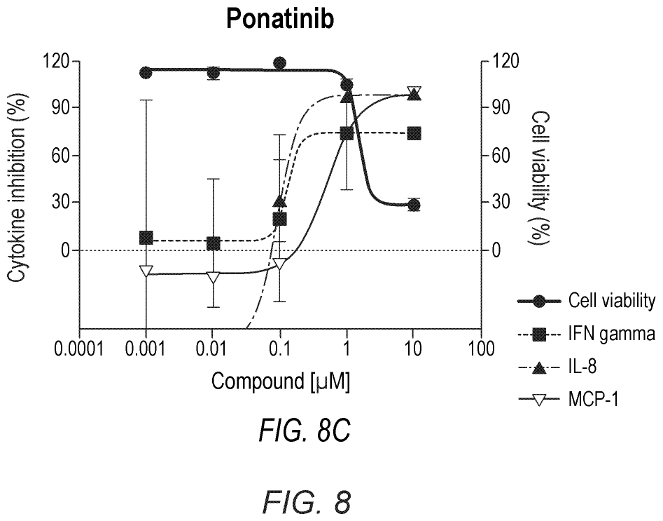

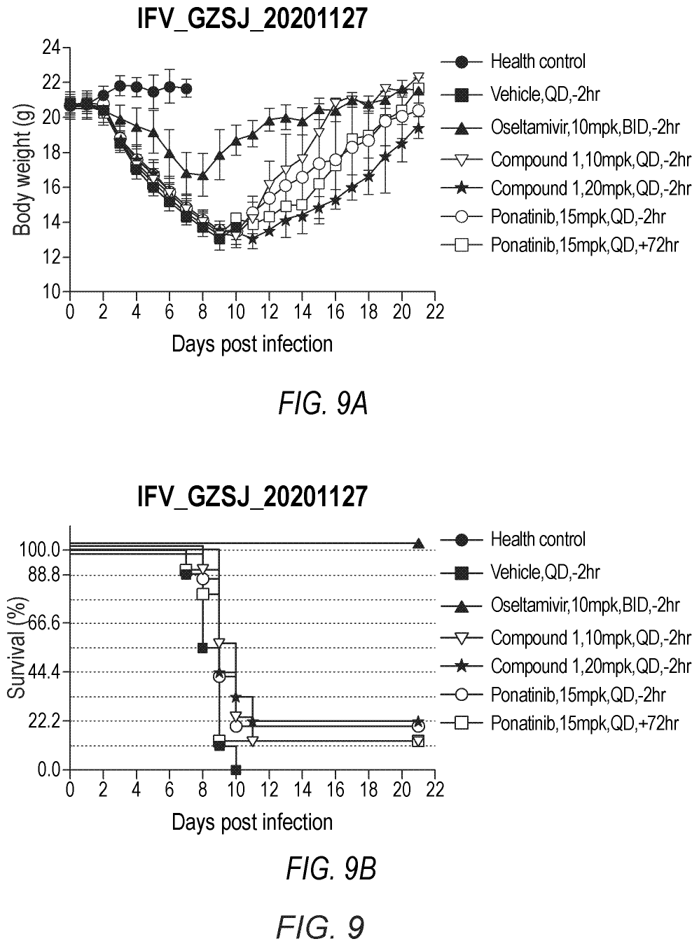

Following the method described in Front Immunol. 2019 Jun. 21; 1393 and Am. J. Physiol., Lung Cell Mol. Physiol. 315: L52-L58, 2018, U937 cells were infected with influenza virus at a MOI of 0.1 and treated with indicated concentrations of Compound 1 and Compound 2. The supernatant of cells was harvested after 24 hours treatment and the production of indicated cytokines were determined using Luminex assay. The method and results are shown below in FIG. 7 and FIGS. 8A-8C. Survival of influenza virus (IFV) infected mice are shown Table 4 and in FIGS. 9A-9B.

| | | Total | Day 21 | | Log-rank |
|---|---|---|---|---|---|
| | Dosing | Animal | Survival | Survival | (Mantel-cox) |
| Study Group | schedule | number | number | rate (%) | test, p value |
| Vehicle control | QD, 2 hr | 9 | 0 | 0 | NA |
| Oseltamivir, | prior to | 9 | 9 | 100 | ****, |
| 10 mpk | infection | | | | p < 0.0001 |
| Compound 1, | | 9 | 1 | 11.11 | *, |
| 10 mpk | | | | | P = 0.0249 |
| Compound 1, | | 9 | 2 | 22.22 | *, |
| 20 mpk | | | | | P = 0.0126 |
| Ponatinib, | | 9 | 2 | 22.22 | *, |
| 15 mpk | | | | | P = 0.0419 |
| Ponatinib, | QD, 72 hr | 9 | 1 | 11.11 | NS, |
| 15 mpk | post | | | | P = 0.2412 |
| | infection | | | | |

As shown in Example 1 and Example 2, Compound 1 inhibits SARS-CoV-2 infection in Vero cell, inhibits the production of cytokines associated with influenza induced cytokine storm, and at the amounts of 5 mg/kg or 10 mg/kg, it improves the survival of influenza virus infected mice.

The Example 1 and Example 2 further indicate that Compound 2 inhibits the production of cytokines associated with influenza induced cytokine storm.

### Example 3 Inhibition of R1PK1 Kinase Assay

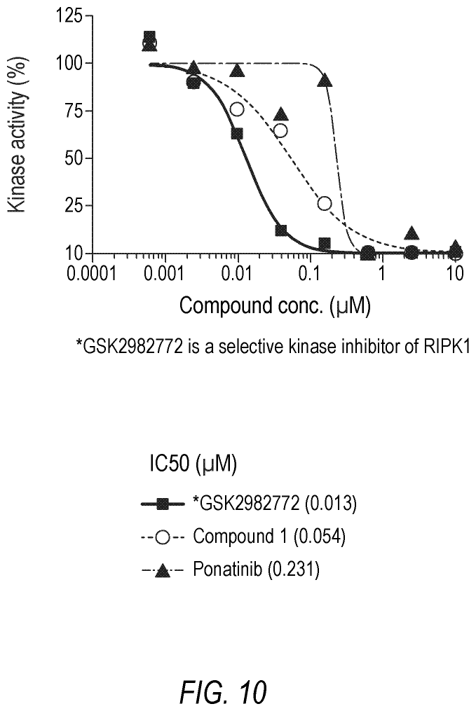

Following the method described in manufacture's instruction of RIPK1 Kinase Enzyme System kit (Promega, V6930), the inhibition effects of Compound 1 and ponatinib on the kinase activity of RIPK1 were determined. GSK2982772, a RIPK1 selective inhibitor, was used as positive control. FIG. 10 shows the assay results. The results demonstrate that Compound 1 inhibits kinase activity of RIPK1 in cell free assay with IC₅₀ value of 54 nM, more potent than ponatinib (IC₅₀ value of 231 nM).

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

We claim:

1. A method of treating a viral infection in a patient in need thereof, comprising administering to the patient a therapeutically effective amount of Compound 1: 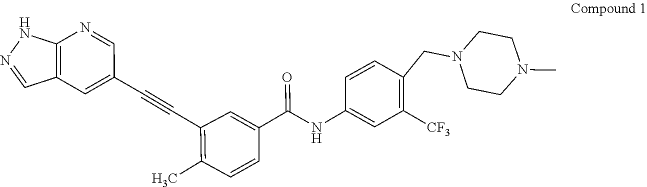 [SMILES: Cc1ccc(C(=O)Nc2ccc(CN3CCN(C)CC3)c(C(F)(F)F)c2)cc1C#Cc1cnc2[nH]ncc2c1]
or a pharmaceutically acceptable salt thereof.

2. The method of claim 1, wherein the viral infection is caused by a coronavirus.

3. The method of claim 2, wherein the viral infection is a coronavirus infection caused by SARS-CoV-2, SARS-CoV, MERS-CoV, 229E, NL63, OC43, or HKUl.

4. The method of claim 3, wherein the coronavirus infection is caused by SARS-CoV-2.

5. The method of any one of claims 1-4, wherein the patient is suffering from COVID-19.

6. The method of claim 3, wherein the coronavirus infection is caused by SARS-CoV.

7. The method of claim 3, wherein the coronavirus infection is caused by MERS-CoV.

8. The method of any one of claims 1-7, wherein the method inhibits a coronavirus polymerase.

9. The method of any one of claims 1-7, wherein the method inhibits an Abelson kinase.

10. The method of any one of claims 1-7, wherein the method inhibits coronaviral membrane fusion.

11. The method of any one of claims 1-7, wherein the method inhibits receptor-interacting serine/threonine-protein kinase 1.

12. The method of any one of claims 1-10, wherein the patient is suffering from cytokine storm syndrome.

13. The method of claim 12, wherein the method inhibits patient's production of cytokines.

14. The method of any one of preceding claims 1-13, wherein Compound 1 or a pharmaceutically acceptable salt thereof is administered orally.

15. The method of any one of preceding claims 1-14, wherein Compound 1 or a pharmaceutically acceptable salt thereof is administered once a day, twice a day, or three times a day.

16. The method of any one of claims 1-15, wherein the therapeutically effective amount is from 0.5 mg to 100 mg, 1 mg to 80 mg, or from 1 mg to 60 mg.

17. The method of any one of claims 1-16, wherein Compound 1 is in a solid dosage form.

18. A pharmaceutical composition for treating a coronavirus infection in a human in need thereof, comprising Compound 1 or a pharmaceutically acceptable salt thereof.

19. Compound 1 or a pharmaceutically acceptable salt or ester thereof, for use in treating a coronavirus infection in a human.

20. Compound 1 or a pharmaceutically acceptable salt or ester thereof, for use in treating SARS-CoV-2 infection in a human.

21. Use of Compound 1 or a pharmaceutically acceptable salt thereof in the manufacture of a medicament for treating a coronavirus infection.

22. A method of treating a viral infection in a patient in need thereof, comprising administering to the patient a therapeutically effective amount of Compound 2: 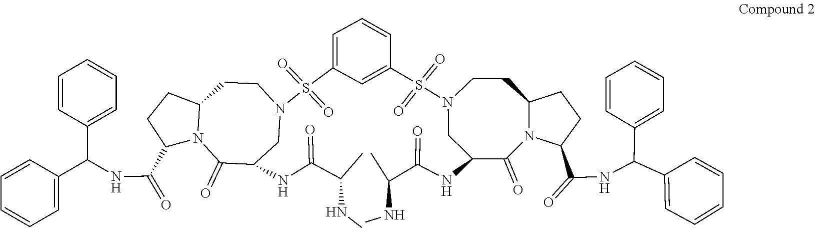
or a pharmaceutically acceptable salt thereof.

23. The method of claim 22, wherein the viral infection is caused by a coronavirus.

24. The method of claim 23, wherein the viral infection is a coronavirus infection caused by SARS-CoV-2, SARS-CoV, MERS-CoV, 229E, NL63, OC43, or HKUl.

25. The method of claim 24, wherein the coronavirus infection is caused by SARS-CoV-2.

26. The method of any one of claims 22-25, wherein the patient is suffering from COVID-19.

27. The method of claim 24, wherein the coronavirus infection is caused by SARS-CoV.

28. The method of claim 24, wherein the coronavirus infection is caused by MERS-CoV.

29. The method of any one of claims 22-28, wherein the method inhibits a coronavirus polymerase.

30. The method of any one of claims 22-28, wherein the method inhibits an Inhibitor of Apoptosis Protein.

31. The method of any one of claims 22-28, wherein the method inhibits a cellular Inhibitor of Apoptosis Protein.

32. The method of any one of claims 22-28, wherein the method selectively induces apoptosis in coronavirus infected cells.

33. The method of any one of claims 22-28, wherein the method achieves coronavirus clearance.

34. The method of any one of preceding claims 22-33, wherein Compound 2 or a pharmaceutically acceptable salt thereof is administered orally.

35. The method of any one of preceding claims 22-34, wherein Compound 2 or a pharmaceutically acceptable salt thereof is administered once a day, twice a day, or three times a day during a treatment cycle.

36. The method of any one of claims 22-34, wherein the therapeutically effective amount is from 0.5 mg to 100 mg, 1 mg to 80 mg, or from 1 mg to 60 mg.

37. The method of any one of claims 22-36, wherein Compound 2 is in a solid dosage form.

38. A pharmaceutical composition for treating a coronavirus infection in a human in need thereof, comprising Compound 2 or pharmaceutically acceptable salt thereof.

39. Compound 2 or a pharmaceutically acceptable salt or ester thereof, for use in treating a coronavirus infection in a human.

40. Compound 2 or a pharmaceutically acceptable salt or ester thereof for use in treating SARS-CoV-2 infection in a human.

41. Use of Compound 2 or a pharmaceutically acceptable salt thereof in the manufacture of a medicament for treating a coronavirus infection.

42. A method of treating a viral infection in a patient in need thereof, comprising administering to the patient a therapeutically effective amount of Compound 3: 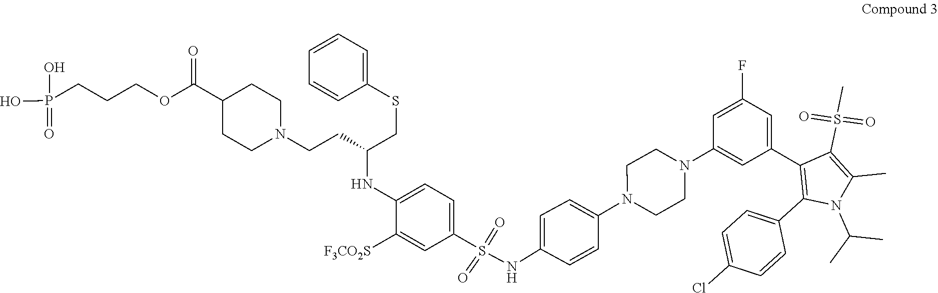
or a pharmaceutically acceptable salt thereof.

43. A method of treating a viral infection in a patient in need thereof, comprising administering to the patient a therapeutically effective amount of Compound 4: 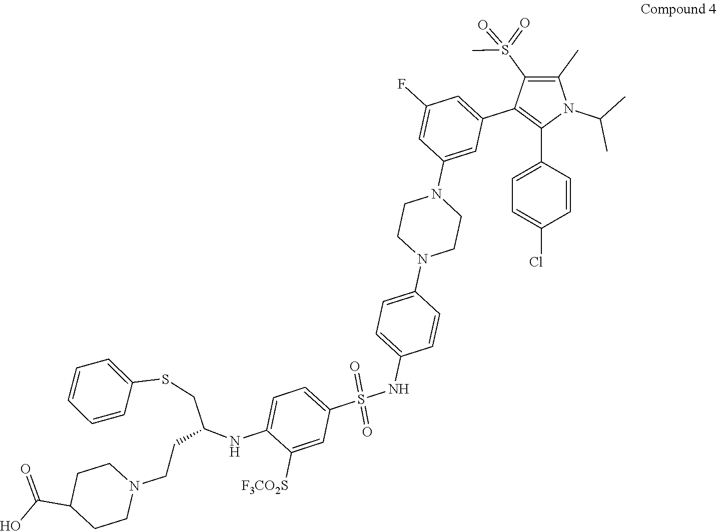
or a pharmaceutically acceptable salt thereof.

44. A method of treating a viral infection in a patient in need thereof, comprising administering to the patient a therapeutically effective amount of Compound 5: 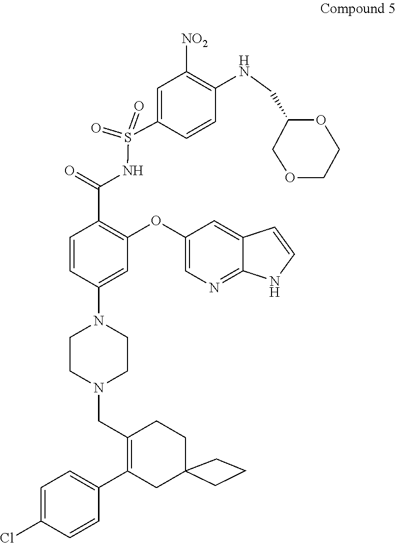 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]
or a pharmaceutically acceptable salt thereof.

45. The method of any one of claims 42-44, wherein the viral infection is caused by a coronavirus.

46. The method of claim 45, wherein the viral infection is a coronavirus infection caused by SARS-CoV-2, SARS-CoV, MERS-CoV, 229E, NL63, OC43, or HKUl.

47. The method of claim 46, wherein the coronavirus infection is caused by SARS-CoV-2.

48. The method of any one of claims 42-47, wherein the patient is suffering from COVID-19.

49. The method of claim 46, wherein the coronavirus infection is caused by SARS-CoV.

50. The method of claim 46, wherein the coronavirus infection is caused by MERS-CoV.

51. The method of any one of claims 42-50, wherein the method inhibits a coronavirus polymerase.

52. The method of any one of claims 42-50, wherein the method inhibits a BCL-2 protein.

53. The method of any one of claims 42-50, wherein the method inhibits a BCL-xL protein.

54. The method of any one of claims 42-50, wherein the method selectively induces apoptosis in coronavirus infected cells.

55. The method of any one of preceding claims 42-54, wherein Compound 3, Compound 4, or Compound 5, or a pharmaceutically acceptable salt thereof is administered orally.

56. The method of any one of preceding claims 42-55, wherein Compound 3, Compound 4, or Compound 5, or a pharmaceutically acceptable salt thereof is administered once a day, twice a day, or three times a day during a treatment cycle.

57. The method of any one of claims 42-56, wherein the therapeutically effective amount is from 0.5 mg to 100 mg, from 1 mg to 80 mg, or from 1 mg to 60 mg.

58. The method of any one of claims 42-57, wherein Compound 3, Compound 4, or Compound 5 is in a solid dosage form.

59. A pharmaceutical composition for treating a coronavirus infection in a human in need thereof, comprising Compound 3, Compound 4, or Compound 5, or a pharmaceutically acceptable salt thereof.

60. Compound 3 or a pharmaceutically acceptable salt or ester thereof for use in treating a coronavirus infection in a human.

61. Compound 4 or a pharmaceutically acceptable salt or ester thereof, for use in treating a coronavirus infection in a human.

62. Compound 5 or a pharmaceutically acceptable salt or ester thereof, for use in treating a coronavirus infection in a human.

63. Compound 3 or a pharmaceutically acceptable salt or ester thereof, for use in treating SARS-CoV-2 infection in a human.

64. Compound 4 or a pharmaceutically acceptable salt or ester thereof, for use in treating SARS-CoV-2 infection in a human.

65. Compound 5 or a pharmaceutically acceptable salt or ester thereof, for use in treating SARS-CoV-2 infection in a human.

66. Use of Compound 3 or a pharmaceutically acceptable salt thereof in the manufacture of a medicament for treating a coronavirus infection.

67. Use of Compound 4 or a pharmaceutically acceptable salt thereof in the manufacture of a medicament for treating a coronavirus infection.

68. Use of Compound 5 or a pharmaceutically acceptable salt thereof in the manufacture of a medicament for treating a coronavirus infection.
